(19) United States
(12) Patent Application Publication
(10) Pub. No.: US 2022/0411448 A1
(43) Pub. Date: Dec. 29, 2022

(54) CHROMENE-4-ONE DERIVATIVES AS BRAIN-DERIVED NEUROTROPHIC FACTOR (BDNF) MIMETICS

(71) Applicant: FLORATEK PHARMA AG, Aubonne (CH)

(72) Inventor: Dan Florin STOICESCU, Aubonne (CH)

(21) Appl. No.: 17/761,270

(22) PCT Filed: Sep. 21, 2020
(86) PCT No.: PCT/EP2020/076213

§ 371 Date: Mar. 17, 2022

(30) Foreign Application Priority Data
Sep. 19, 2019 (GB) 1913548.2

(51) Int. Cl. C07F 9/54

(52) U.S. Cl. CPC C07F 9/5456

(57) ABSTRACT

The present invention relates to chromen-4-one derivatives comprising a quaternary group, and to associated multi-salts, solvates, prodrugs and pharmaceutical compositions. The present invention also relates to the use of such compounds and compositions in the treatment and prevention of medical disorders and diseases, most especially by modulation of neurotrophic factors (such as BDNF) pathways and modulation of mitochondrial function Formula (I).

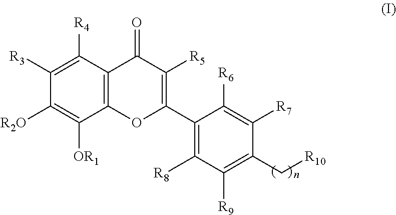

### FIELD OF THE INVENTION

The present invention relates to chromen-4-one derivatives comprising a lipophilic cation, and to associated multi-salts, solvates, prodrugs and pharmaceutical compositions. The present invention also relates to the use of such compounds and compositions in the treatment and prevention of medical disorders and diseases, most especially those related to neurotrophic factors pathways and mitochondrial activity.

### BACKGROUND

Neurotrophic factors are endogenous soluble proteins that regulate the cell cycle, growth, differentiation, and survival of neurons [Barde Y.-A. (1990) The nerve growth factor family. Prog. Growth Factor Res. 2:237-348]. Members of the neurotrophic family include nerve growth factor (NGF), brain-derived neurotrophic factor (BDNF), glial-derived neurotrophic factor (GDNF), neurotrophin-3 (NT-3), and neurotrophin-4 (NT-4).

Brain-derived neurotrophic factor (BDNF), as a member of neurotropic family in nervous system, has various therapeutic effects via activating tyrosine kinase (TrkB). The Trk receptors are glycoproteins that have a molecular weight in the range of 140-145 kDa. Each neurotrophin appears to bind to a unique isoform of the Trk receptors. For example, NGF has a greater specificity to bind to the TrkA receptor, NT-3 interacts with TrkC, and both BDNF and NT-4 bind to TrkB [Reichardt, L. F. 2006, Neurotrophin-regulated signalling pathways. Philos. Trans. R. Soc. Lond. B Biol. Sci. 361, 1545-1564]. Extracellular BDNF binds to TrkB receptors and causes receptor dimerization, which leads to phosphorylation of tyrosine residues within the cytoplasm and activates kinases.

BDNF has however poor delivery and short half-life in vivo which hamper its clinical usefulness [Deng P, Engineered BDNF producing cells as a potential treatment for neurologic disease. Expert Opin Biol Ther. 2016; 16(8):1025-1033]. 7,8-dihydroxyflavone (7,8-DHF) has been discovered as a promising small molecular TrkB agonist which fully mimics the physiological properties of BDNF [Liu C, 7,8-dihydroxyflavone, a small molecular TrkB agonist, is useful for treating various BDNF-implicated human disorders. Transl Neurodegener. 2016. 5:2]. 7,8-DHF has been reported to be useful in improving cognitive impairment in many diseases, such as Alzheimer disease (AD) [Zhang Z, 7,8-Dihydroxyflavone Prevents Synaptic Loss and Memory Deficits in a Mouse Model of Alzheimer's Disease. Neuropsychopharmacology (2014) 39, 638-650], ameliorating nigrostriatal dopaminergic neurons loss and damage to striatal fibers in the MPTP-induced PD [Nie S, 7,8-Dihydroxyflavone Protects Nigrostriatal Dopaminergic Neurons from Rotenone-Induced Neurotoxicity in Rodents. Parkinson's Disease Volume 2019], enhancing brain plasticity and memory formation [Krishna G, 7,8-Dihydroxyflavone facilitates the action exercise to restore plasticity and functionality: Implications for early brain trauma recovery. Biochimica et Biophysica Acta (BBA)—Molecular Basis of Disease Volume 1863, Issue 6, June 2017, 20 Pages 1204-1213].

Because 7,8-DHF has only modest oral bioavailability and a moderate pharmacokinetic (PK) profile, a number of prodrugs and derivatives have been developed [Chen C, The prodrug of 7,8-dihydroxyflavone development and therapeutic efficacy for treating Alzheimer's disease PNAS Jan. 16, 2018 115 (3) 578-583].

While BDNF mimetics have the potential to ameliorate a number of neurological conditions, in recent years the role of mitochondria has been increasingly studied and a large number of studies have indicated the possible pathogenic role of mitochondria in neurological diseases and the possible benefits of targeting drugs to modulate mitochondrial activity [Kumar A. Editorial (Thematic Selection: Mitochondrial Dysfunction &Neurological Disorders). Curr Neuropharmacol. 2016; 14(6):565-566]. Mitochondria are critical regulators of cell death, a key feature of neurodegeneration. Mutations in mitochondrial DNA and oxidative stress both contribute to ageing, which is the greatest risk factor for neurodegenerative diseases [Arun S, Mitochondrial Biology and Neurological Diseases. Curr Neuropharmacol. 2016 February; 14(2): 143-154]. Impaired Ca influx, energy supply, control of apoptosis by mitochondria or increased ROS (reactive oxygen species) production can contribute to the progressive decline of long-lived postmitotic cells, such as neurons. Furthermore, mitochondrial ROS generation is known as key factors accountable for cell death and disease progression in age-dependent diseases [Reddy P H, Mitochondria as a therapeutic target for aging and neurodegenerative diseases. Curr. Alzheimer Res 2011, 8: 393-409]. As critical regulators and potential cause of neurological conditions, mitochondria appear as critical drug targeting organelles in the brain cellular environment.

Over a century ago, it was recognized that the brain milieu contains large numbers of glia cells intimately associated with neurons. However, only recently many studies showed that glia not only support a number of essential neuronal functions, but also actively communicate with neurons and with one another. By doing so, glia influence nervous system functions that have long been thought to be strictly under neuronal control [Stevens B. Glia: Current Biology. 2003 Vol 13 No 12. Pages R469-R472]. As glia are a major source of trophic factors, it is not surprising that they are proposed to be critical regulators of neuronal migration, growth and survival during development-consistent with their well-accepted support role.

Other glial roles that are well-established include maintaining the ionic milieu of nerve cells, modulating the rate of nerve signal propagation, modulating synaptic action by controlling the uptake of neurotransmitters, providing a scaffold for some aspects of neural development, and aiding in (or preventing, in some instances) recovery from neural injury [Zuchero J B, Glia in mammalian development and disease. Development 2015 142: 3805-3809]. There are three types of glial cells in the mature central nervous system: astrocytes, oligodendrocytes, and microglial cells. The major function of astrocytes is to maintain, in a variety of ways, an appropriate chemical environment for neuronal signaling.

While astrocytes respond to increases in neuronal activity and metabolic demand by upregulating glycolysis and glycogenolysis, astrocytes also possess significant capacity for oxidative (mitochondrial) metabolism. Mitochondria mediate energy supply and metabolism, cellular survival, ionic homeostasis, and proliferation [Jackson J G, Regulation of mitochondrial dynamics in astrocytes: Mechanisms, consequences, and unknowns. Glia. 2018 June; 66(6):1213-1234].

It is only relatively recent that it is becoming clear that the dysfunction of astrocytes, the so called “reactive astrogliosis,” is associated with all neurodegenerative diseases including AD, and characterized with various complex molecular and functional changes in the cells [Osborn L M, Kamphuis W, Wadman W J, Hol E M. Astrogliosis: An integral player in the pathogenesis of Alzheimer's disease. Prog Neurobiol. 2016; 144:121-141]. It has also been previously shown that many of astrocytes dysfunctions is largely due to mitochondrial dynamics.

Interestingly, mitochondrial dysfunction is a key pathological feature of AD and precedes Aβ plaque deposition [Yao J, Mitochondrial bioenergetic deficit precedes Alzheimer's pathology in female mouse model of Alzheimer's disease. Proceedings of the National Academy of Sciences of the United States of America. 2009; 106(34):14670-14675] and is accompanied by a progressive reduction of the cerebral metabolic rates of glucose. Thus, several new therapeutic approaches have tested the efficacy of mitochondria-targeted molecules in delaying AD progression [Wilkins H M, New therapeutics to modulate mitochondrial function in neurodegenerative disorders. Current Pharmaceutical Design. 2017; 23(5):731-752].

There is a need to provide compounds with improved pharmacological and/or physiological and/or physiochemical properties and/or those that provide a useful alternative to known compounds.

### SUMMARY OF THE INVENTION

The present invention addresses the limitations of current BDNF small molecules mimetics with design features aimed at increasing the brain blood barrier penetration, longer half-life in circulation and therefore better pharmacokinetic profile. In addition, the series of compounds represent a novel class of mitochondria targeted compounds. Without wishing to be bound by theory, the compounds are effective because of the presence of a lipophilic ion. Additionally or alternatively, the discovered compound series optimizes the alkyl linker used to connect the lipophilic ion with the biologically active moiety. It is envisioned that this novel series will exert the dual effect of a neurotrophic factor mimetic and mitochondria modulator, thus acting in both neurons and astrocytes with potential beneficial effects on many disorders, e.g. neurodegenerative disorders.

A first aspect of the invention provides a compound of formula (I):

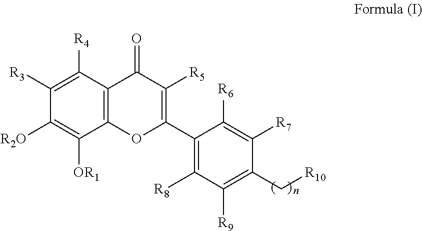

wherein:
R¹ and R², independently, are selected from H, hydroxyl protecting groups, —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, and —OC(C≡CH)H₂;
or R¹ and R² together form a C₁₋₃ alkylene group;
R³, R⁴, R⁵, R⁶, R⁷, R⁸, and R⁹, independently, are selected from H; halo; —CN; —NO₂; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and —OCOR^{β}; each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X rhodamine B X, rhodamine 6G X, rhodamine 19 X, or rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
and
n is an integer from 1 to 14.

In one embodiment, R¹ and R² are independently selected from H and hydroxyl protecting groups.

In one embodiment, R¹ and R² are independently selected from H and hydroxyl protecting groups; or R¹ and R² together form a C₁₋₃ alkylene group.

In one embodiment, R¹ and R² are independently selected from H—CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, and —OC(C≡CH)H₂.

In one embodiment, R¹ and R² are independently selected from H, —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, and —OC(C≡CH)H₂, or R¹ and R² together form a C₁₋₃ alkylene group.

In one embodiment, R¹ and R² are H.

In one embodiment, R³, R⁴, R⁵, R⁶, R⁷, R⁸, and R⁹ are independently selected from H; halo; —CN; —NO₂; —R^{β}; —OH; —OR^{β}; —NH₂; —NHR^{β}; —N(R¹)₂; —CHO; —COR^{β}; —COOH; —COOR^{β}; and —OCOR^{β}.

In one embodiment, R³, R⁴, R⁵, R⁶, R⁷, R⁸, and R⁹ are H.

In one embodiment, R^{β} is independently selected from a C₁-C₆ alkyl, C₂-C₆ alkenyl, C₂-C₆ alkynyl or C₃-C₁₄ cyclic group, and wherein any —R³ may optionally be substituted with one or more halo, —OH, —NH₂, —CN, —NO₂, —C≡CH, —CHO, —CON(CH₃)₂ or oxo (═O) groups.

In one embodiment, R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, rhodamine 6G X, rhodamine 19 X, or rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group.

In one embodiment, R¹⁰ is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups.

In one embodiment, each R¹¹ group is the same; preferably each R¹¹ is a phenyl group.

In one embodiment, the counter anion X is fluoride, chloride, bromide or iodide.

A second aspect of the invention provides a compound selected from the group consisting of:

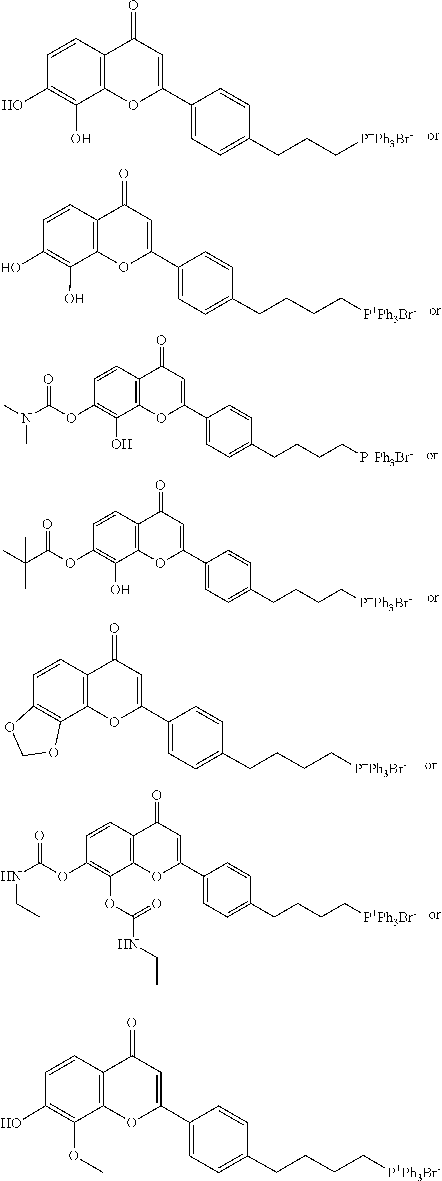

A third aspect of the invention provides pharmaceutically acceptable multi-salt, solvate or prodrug of the compound of the first or second aspect of the invention.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. In one embodiment, the disease, disorder or condition is a central nervous system disease.

An sixth aspect of the invention provides the use of a compound of the first or second aspect, a pharmaceutically effective multi-salt, solvate or prodrug of the third aspect, or a pharmaceutical composition according to the fourth aspect, in the manufacture of a medicament for the treatment or prevention of a disease, disorder or condition. Typically the treatment or prevention comprises the administration of the compound, multi-salt, solvate, prodrug or pharmaceutical composition to a subject. In one embodiment, the disease, disorder or condition is a central nervous system disease.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is a central nervous system disease.

An eighth aspect of the invention provides a method of modulating neurotrophic factors pathways (such as BDNF pathways), the method comprising the use of compound of the first or second aspect of the invention, or a pharmaceutically acceptable multi-salt, solvate or prodrug of the third aspect of the invention, or a pharmaceutical composition of the fourth aspect of the invention, to modulate neurotrophic factors pathways (such as BDNF pathways).

A ninth aspect of the invention provides a method of modulating mitochondrial function, the method comprising the use of compound of the first or second aspect of the invention, or a pharmaceutically acceptable multi-salt, solvate or prodrug of the third aspect of the invention, or a pharmaceutical composition of the fourth aspect of the invention, to modulate mitochondrial function.

### Definitions

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “haloalkyl” substituent group or haloalkyl group in a substituent group refers to an alkyl, alkenyl, or alkynyl substituent group or moiety including one or more carbon atoms and one or more halo atoms, e.g. Cl, Br, I, or F. Each halo atom replaces a hydrogen of the alkyl, alkenyl, or alkynyl substituent group or moiety. Examples include —CH₂F—CHF₂, —CHI₂, —CHBr₂, —CHCl₂, —CF₃, —CH₂CF₃ and CF₂CH₃.

An “alkoxy” substituent group or alkoxy group in a substituent group refers to an alkyl, alkenyl, or alkynyl substituent group or moiety including one or more carbon atoms and one or more oxygen atoms. Each oxygen atom replaces a carbon atom (for example the terminal or bonding carbon) of the alkyl, alkenyl, or alkynyl substituent group or moiety. Examples include —OCH₃, —OCH₂CH₃, —OCH₂CH₂CH₃, and —OCH(CH₃)(CH₃).

An “alkylthio” substituent group or alkylthio group in a substituent group refers to an alkyl, alkenyl, or alkynyl substituent group or moiety including one or more carbon atoms and one or more sulphur atoms. Each sulphur atom replaces a carbon atom (for example the terminal or bonding carbon) of the alkyl, alkenyl, or alkynyl substituent group or moiety. Examples include —SCH₃, —SCH₂CH₃, —SCH₂CH₂CH₃, and —SCH(CH₃)(CH₃).

An “alkylsulfinyl” substituent group or alkylsulfinyl group in a substituent group refers to an alkyl, alkenyl, or alkynyl substituent group or moiety including one or more carbon atoms and one or more sulfinyl groups (—S(═O)—). Each sulfinyl group replaces a carbon atom (for example the terminal or bonding carbon) of the alkyl, alkenyl, or alkynyl substituent group or moiety. Examples include —S(═O)CH₃, —S(═O)CH₂CH₃, —S(═O)CH₂CH₂CH₃, and —S(═O)CH(CH₃)(CH₃).

An “alkylsulfonyl” substituent group or alkylsulfonyl group in a substituent group refers to an alkyl, alkenyl, or alkynyl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

An “arylsulfonyl” substituent group or arylsulfonyl group in a substituent group refers to an aryl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

A “cyclic” substituent group or a cyclic moiety in a substituent group refers to any hydrocarbyl ring, wherein the hydrocarbyl ring may be saturated or unsaturated and may include one or more heteroatoms, e.g. N, O or S, in its carbon skeleton. Examples of cyclic groups include aliphatic cyclic, cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl and thiomorpholinyl groups.

An “aliphatic cyclic” substituent group or aliphatic cyclic moiety in a substituent group refers to a hydrocarbyl cyclic group or moiety that is not aromatic. The aliphatic cyclic group may be saturated or unsaturated and may include one or more heteroatoms, e.g. N, O or S, in its carbon skeleton. Examples include cyclopropyl, cyclohexyl and morpholinyl. Unless stated otherwise, an aliphatic cyclic substituent group or moiety may include monocyclic, bicyclic or polycyclic hydrocarbyl rings.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

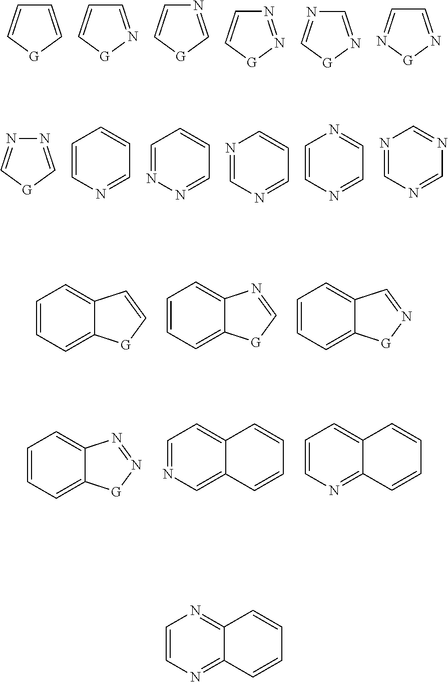

wherein G═O, S or NH.

For the purposes of the present specification, rhodamine B is a group of either Formula A or Formula B:

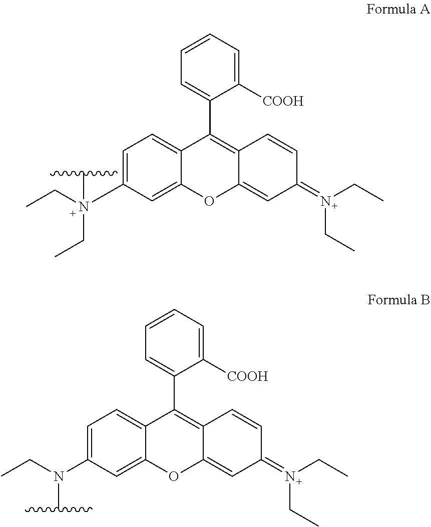

For the purposes of the present specification, rhodamine 6G is a group of the following formula:

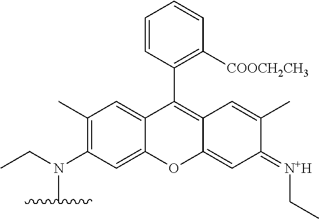

For the purposes of the present specification, rhodamine 19 is a group of the following formula:

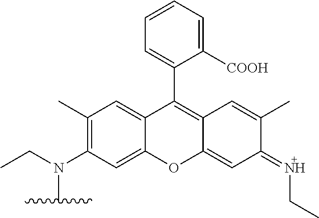 [SMILES: *N(CC)c1cc2oc3c/c(=N\CC)c(C)cc-3c(-c3ccccc3C(=O)O)c2cc1C]

For the purposes of the present specification, rhodamine 123 is a group of the following formula:

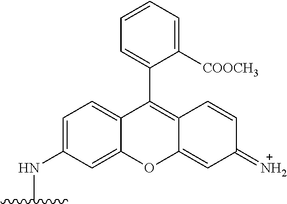 [SMILES: *Nc1ccc2c(-c3ccccc3C(=O)OC)c3ccc(=[NH2+])cc-3oc2c1]

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

Typically a substituted group comprises 1, 2, 3 or 4 substituents, more typically 1, 2 or 3 substituents, more typically 1 or 2 substituents, and even more typically 1 substituent.

Unless stated otherwise, any divalent bridging substituent (e.g. —O—, —S—, —NH—, —N(R^{β})— or —R^{α}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Where reference is made to a carbon atom of a group being replaced by an N, O or S atom, what is intended is that:

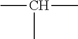

is replaced by

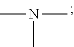

—CH₂— is replaced by —NH—, —O— or —S—;
—CH₃ is replaced by —NH₂, —OH, or —SH;
—CH═ is replaced by —N═;
CH₂═ is replaced by NH═, O═ or S═; or
CH≡ is replaced by N≡.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are counted as carbon atoms when calculating the number of carbon atoms in a Cₓ-C_{y} group. For example, a morpholinyl group is to be considered a C₆ heterocyclic group, not a C₄ heterocyclic group.

A “protecting group” refers to a grouping of atoms that when attached to a reactive functional group (e.g. OH) in a compound masks, reduces or prevents reactivity of the functional group.

In the context of the present specification, ═ is a double bond; ≡ is a triple bond.

The protection and deprotection of functional groups is described in ‘Protective Groups in Organic Synthesis’, 2^{nd} edition, T. W. Greene and P. G. M Wuts, Wiley-Interscience.

### BRIEF DESCRIPTION OF FIGURES

FIG. 1 is a graph showing cellular viability for different concentrations of a compound of the invention.

FIG. 2 is a graph showing glucose uptake following application of a compound of the invention to a cell culture.

FIG. 3 is a graph showing lactate release following application of different concentrations of a compound of the invention to a cell culture.

FIG. 4 is a graph showing reactive oxidation species (ROS) formation following application of different concentrations of a compound of the invention to a cell culture.

FIG. 5 is a graph showing ATP/ADP ratio following application of different concentrations of a compound of the invention to a cell culture.

FIG. 6 is a graph showing NAD/NAHD ratio following application of different concentrations of a compound of the invention to a cell culture.

FIG. 7 shows four graphs showing the effect of a compound of the invention on the mRNA expression of genes related to plasticity (Arc, cFos, and Zif268) and Cox2. In the graphs of FIG. 7, 1=Vehicle control; 2=Compound A 10 μM treatment 1 h; 3=Compound A 10 μM treatment 2 h; 4=Compound A 1 μM treatment 1 h; and 5=Compound A 1 μM treatment 2 h.

In the figures, * refers to a statistical significance (p)≤0.1; ** refers to a statistical significance (p)≤0.05; and *** refers to a statistical significance (p)≤0.001.

FIG. 8 shows the maximum peak of calcium kinetic when neurons are treated with 10 μm glutamate in the presence of various concentrations of SND135.

FIG. 9 shows the maximum peak of calcium kinetic when neurons are treated with 30 μm glutamate in the presence of various concentrations of SND135.

FIG. 10 shows that glutamate increases mitochondria potential, which is restored by the control compound [(+)-5-methyl-10,11-dihydroxy-5H-dibenzo(a,d)cyclohepten-5,01-imine] also known as dizocilpine hydrogen maleate (MK801).

FIG. 11 shows the effect of SND135 on the mitochondria potential at a concentration of glutamate of 10 μM in comparison to vehicle.

FIG. 12 shows the effect of SND135 on the mitochondria potential at a concentration of glutamate of 30 μM in comparison to vehicle.

FIG. 13 shows the effect of SND135 on the mitochondria potential at a concentration of glutamate of 100 μM in comparison to vehicle.

FIG. 14 shows that SND118 and SND124 restore mitochondria membrane potential (MMP) decreased by iodoacetic acid (IAA) lesion. VC=vehicle control; LC=lesion control.

FIG. 15 shows that SND118 and SND124 increase cell survival upon IAA lesion.

FIG. 16 shows the effect of SND118 on MPP+ induced apoptosis. VC=vehicle control; LC=lesion control.

FIG. 17 shows the effect of SND118 on MPP+ induced reactive oxygen species (ROS). VC=vehicle control; LC=lesion control.

FIGS. 18-20 show measurement of inflammatory cytokines and NO in BV2 cell line in the presence of test and control treatment.

### DETAILED DESCRIPTION

A first aspect of the invention provides a compound of formula (I):

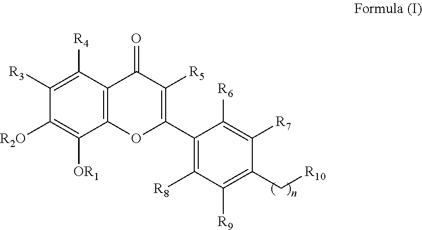

wherein:
R¹ and R², independently, are selected from H, hydroxyl protecting groups, —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, —OC(C≡CH)H₂; or R¹ and R² together form a C₁₋₄ alkylene group;
R³, R⁴, R⁵, R⁶, R⁷, R⁸, and R⁹, independently, are selected from H; halo; —CN; —NO₂; —R^{β}; —OH; —OR^{β}; —SH; —SR; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and —OCOR^{β}; each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
and
n is an integer from 1 to 14.

In one embodiment, R¹ and R², independently, are selected from H, hydroxyl protecting groups, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, —OC(C≡CH)H₂.

In one embodiment, R¹ and R² together form a C₁₋₄ alkylene group.

In one embodiment, R¹ and R² are independently selected from H and hydroxyl protecting groups.

In one embodiment, R¹ and R² are independently selected from H, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, and —OC(C≡CH)H₂.

In one embodiment, R¹ and R² are independently selected from H, —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, and —OC(C≡CH)H₂, or R¹ and R² together form a C₁₋₄ alkylene group.

In one embodiment, R¹ and R² are independently selected from H, —C(O)R¹³, —C(O)OR¹³, —C(O)NHR¹³, —C(O)N(R¹³)₂, —OCF₃, —OCHF₂, and —OC(C≡CH)H₂.

In one embodiment, R¹ and R² are independently selected from H, —C₁₋₄ alkyl, —C(O)R¹³, —C(O)OR¹³, —C(O)NHR¹³, —C(O)N(R¹³)₂, —OCF₃, —OCHF₂, and —OC(C≡CH)H₂, or R¹ and R² together form a C₁₋₄ alkylene group.

In one embodiment, R¹ and R² are independently selected from H, —C(O)R¹³, —C(O)NHR¹³, and —C(O)N(R¹³)₂.

In one embodiment, R¹ and R² are independently selected from H, —C₁₋₄ alkyl, —C(O)R¹³, —C(O)NHR¹³, and —C(O)N(R¹³)₂, or R¹ and R² together form a C₁₋₄ alkylene group.

In one embodiment, R¹ and R² are independently selected from H, —CO^{t}Bu, —CONHCH₃, —CONHCH₂CH₃ and —CON(CH₃)₂.

In one embodiment, R¹ and R² are independently selected from H, -Me, —CO^{t}Bu, —CONHCH₃, —CONHCH₂CH₃ and —CON(CH₃)₂; or R¹ and R² together form a methylene group.

In one embodiment, R¹ and R² are the same. For example, R¹ and R² are both H. Alternatively, R¹ and R² are both —C(O)NHR¹³. In one embodiment, R¹ is H. In one embodiment, R² is H. In one embodiment, R¹ and R² are H.

In one embodiment, R¹ and R² are different. For example, R₁ may be —H or C₁₋₄ alkyl, and R₂ is selected from —C(O)R¹³, —C(O)NHR¹³, and —C(O)N(R¹³)₂.

In one embodiment, R³, R⁴, R⁵, R⁶, R⁷, R⁸, and R⁹, independently, are selected from H; halo; —CN; —NO₂; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(RD)₂; —CHO; —COR^{β}; —COOH; —COOR^{β}; and —OCOR^{β}.

In one embodiment, R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; —N(RD)₂; —CHO; —COR^{β}; —COOH; —COOR^{β}; and —OCOR^{β}.

In one embodiment, R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; and —N(R^{β})₂.

In one embodiment, R³, R⁴, R⁵, R⁶, R⁷, R⁸, and R⁹ independently, are selected from H; halo; —CN; —NO₂; —R^{β}; —OH; —OR^{β}; and —NH₂.

In one embodiment, R³, R⁴, R⁵, R⁶, R⁷, R⁸, and R⁹ are each H.

In one embodiment, R³, R⁴, R⁵, R⁶, R⁷, R⁸, and R⁹ are the same.

In one embodiment, each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In one embodiment, each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more halo, —OH, —NH₂, —CN, —NO₂, —C≡CH, —CHO, —CON(CH₃)₂ or oxo (═O) groups.

In one embodiment, each —R^{β} is independently selected from a C₁-C₆ alkyl, C₂-C₆ alkenyl, C₂-C₆ alkynyl or C₃-C₁₄ cyclic group.

In one embodiment, each —R^{β} is independently selected from —CF₃ and —CHF₂.

In one embodiment, each —R^{β} is independently selected from a methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R^{β} is independently selected from a methyl, ethyl, n-propyl, i-propyl, n-butyl, i-butyl, t-butyl, or n-pentyl group.

In one embodiment, X is selected from but not limited to halides (for example fluoride, chloride, bromide or iodide) or other inorganic anions (for example nitrate, perchlorate, sulfate, bisulfate, or phosphate) or organic anions (for example propianoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonare, ethanesulfonare, 2-hydroxyethanesulfonate, benzenesulfonate, toluene-p-sulfonate, naphthalene-2-sulfonate, camphorsulfonate, ornithinate, glutamate or aspartate).

In one embodiment, X may be a fluoride, chloride, bromide or iodide.

In one embodiment, X is bromide or chloride.

In one embodiment, X is bromide.

In one embodiment, R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion. For example, X may be bromide or chloride.

In one embodiment, R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, R¹⁰ is —[P(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion. For example, X may be bromide or chloride.

In one embodiment, R¹⁰ is —[P(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, R¹⁰ is —[P(R¹¹)₃]X, wherein each —R¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, R¹⁰ is —[P(R¹¹)₃]X, wherein each —R¹¹ is independently selected from H, or C₁-C₆ alkyl, or C₃-C₁₄ aryl group; and wherein X is a counter anion. For example, X may be bromide or chloride.

In one embodiment, R¹⁰ is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups; and wherein X is a counter anion. For example, X may be bromide or chloride.

In one embodiment, two of the R¹¹ groups are the same. In one embodiment, each R¹ group is the same.

In one embodiment, R¹⁰ is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; and wherein X is a counter anion. For example, X may be bromide or chloride.

In one embodiment, each R¹¹ is a phenyl group.

In one embodiment, R¹⁰ is —[P(Ph)₃]X, wherein X is a counter anion. For example, X may be bromide or chloride, or X may be bromide.

In one embodiment, R¹⁰ is —[P(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group; X is a counter anion; and n is an integer from 1 to 6. For example, X may be bromide or chloride.

In one embodiment, R¹⁰ is —[P(R¹¹)₃]X; X is a counter anion; and n is an integer from 1 to 6. For example, X may be bromide or chloride. For example, n may be an integer from 2 to 5.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl.

In one embodiment, each —R¹³ is independently selected from C₁₋₄ alkyl.

In one embodiment, each —R¹³ is independently selected from a H, methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R¹³ is independently selected from a H, methyl, ethyl, n-propyl, i-propyl, n-butyl, i-butyl, t-butyl, or n-pentyl group.

In one embodiment, each —R¹³ is independently selected from H, methyl, ethyl, propyl, and butyl.

In one embodiment, each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;

In one embodiment, each R¹⁴ is independently selected from a halo, —NO₂, —CN, —OH, —NH₂, mercapto, formyl, carboxy, or carbamoyl group.

In one embodiment, each —R¹⁴ is independently selected from methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl.

In one embodiment, each —R¹⁴ is independently selected from a methyl, ethyl, n-propyl, i-propyl, n-butyl, i-butyl, t-butyl, or n-pentyl group.

In one embodiment, each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;

In one embodiment, n is an integer from 1 to 14. In one embodiment, n is an integer from 1 to 6. In one embodiment, n is an integer from 1 to 4. In one embodiment, n is 3, 4 or 5. In one embodiment, n is 3. In one embodiment, n is 4.

In one embodiment, R¹ is H, and R² is selected from —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, —OC(C≡CH)H₂. For example, R² is selected from —C₁₋₄ alkyl, —C(O)R¹³, or —C(O)NHR¹³, —C(O)N(R¹³)₂. For example, R² is selected from —C(O)R¹³, or —C(O)N(R¹³)₂.

In one embodiment, R¹ is selected from —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, —OC(C≡CH)H₂; and R² is H. For example, R¹ is selected from —C₁₋₄ alkyl, —C(O)R¹³, or —C(O)NHR¹³, —C(O)N(R¹³)₂. For example, R¹ is selected from —C₁₋₄ alkyl.

In one embodiment, the invention provides a compound of formula (I), wherein:
R¹ and R² are H;
R³, R⁴, R⁵, R⁶, R⁷, R⁸, and R⁹, independently, are selected from H; halo; —CN; —NO₂; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(RD)₂; —CHO; —COR^{β}; —COOH; —COOR^{β}; and —OCOR^{β}; each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups; R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; X is a counter anion; and n is an integer from 1 to 14.

In one embodiment, R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; —N(RD)₂; —CHO; —COR^{β}; —COOH; —COOR^{β}; and —OCOR^{β}; each —R³ is independently selected from a C₁-C₆ alkyl, C₂-C₆ alkenyl, C₂-C₆ alkynyl or C₃-C₁₄ cyclic group; R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group; X is a counter anion; and n is an integer from 1 to 6. For example, X may be bromide or chloride, or X may be bromide.

In one embodiment, R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; and —N(RD)₂; each —R³ is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹¹ is independently selected from H, or C₁-C₆ alkyl, or C₃-C₁₄ aryl group; and X is a counter anion; and n is an integer from 1 to 6. For example, X may be bromide or chloride, or X may be bromide.

In one embodiment, R³, R⁴, R⁵, R⁶, R⁷, R⁸, and R⁹ independently, are selected from H; halo; —CN; —NO₂; —R; —OH; —OR^{β}; —NH₂; —NHR^{β}; and —N(RD)₂; each —R^{β} is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹¹ is independently selected from a C₃-C₁₄ aryl group; wherein any —R¹¹ may optionally be substituted with one or more C₁-C₄ alkyl, halo, —OH, —NH₂, —CN, —C≡CH or oxo (═O) groups; X is a counter anion; and n is an integer from 1 to 6. For example, X may be bromide or chloride, or X may be bromide.

In one embodiment, R³, R⁴, R⁵, R⁶, R⁷, R⁸, and R⁹ independently, are selected from H; halo; —CN; —NO₂; —R^{β}; —OH; —OR^{β}; and —NH₂; each —R^{β} is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is an integer from 1 to 6. For example, X may be bromide or chloride, or X may be bromide.

In one embodiment, R³, R⁴, R⁵, R⁶, R⁷, R⁸, and R⁹ are each H; R¹⁰ is —[P(R¹¹)₃]X, wherein each —R¹¹ is a phenyl group; each phenyl may group optionally be substituted with one or more C₁-C₄ alkyl, halo, —OH, —NH₂, —CN, —C≡CH or oxo (═O) groups; X is a counter anion; and n is an integer from 1 to 4. For example, X may be bromide or chloride, or X may be bromide.

In one embodiment, R³, R⁴, R⁵, R⁶, R⁷, R⁸, and R⁹ are each H; R¹⁰ is —[P(Ph)₃]X; X is a counter anion; and n is an integer from 1 to 4. For example, X may be bromide or chloride, or X may be bromide.

In one embodiment, the compound of formula (I) is:

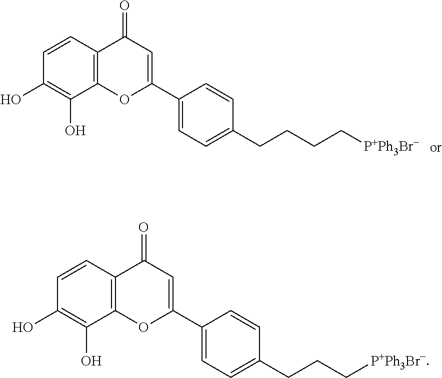

In one embodiment, the compound of formula (I) is:

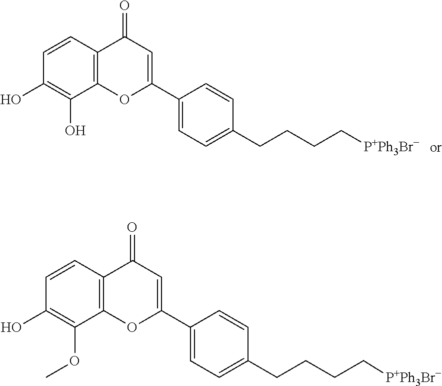

In one embodiment, the invention provides a compound of formula (I), wherein:

R¹ and R² are hydroxyl protecting groups;
R³, R⁴, R⁵, R⁶, R⁷, R⁸, and R⁹, independently, are selected from H; halo; —CN; —NO₂; —R^{β}; —OH; —OR^{β}; —SH; —SR; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and —OCOR^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; X is a counter anion;
and
n is an integer from 1 to 14.

In one embodiment, R¹ and R² are hydroxyl protecting groups; R³, R⁴, R⁵, R⁶, R⁷, R⁸, and R⁹ independently, are selected from H; halo; —CN; —NO₂; —Rn; —OH; —OR^{β}; —NH₂; —NHR³; —N(R¹)₂; —CHO; —COR³; —COOH; —COOR³; and —OCOR³; each —R³ is independently selected from a C₁-C₆ alkyl, C₂-C₆ alkenyl, C₂-C₆ alkynyl or C₃-C₁₄ cyclic group; R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹ is independently selected from H, C₁-C₆ alkyl, C₂-C₆ alkenyl, C₃-C₁₄ aryl group, or C₃-C₁₄ aliphatic cyclic group; X is a counter anion; and n is an integer from 1 to 6. For example, X may be bromide or chloride, or X may be bromide.

In one embodiment, R¹ and R² are hydroxyl protecting groups; R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; and —N(R^{β})₂; each —R^{β} is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹ is independently selected from H, or C₁-C₆ alkyl, or C₃-C₁₄ aryl group; and X is a counter anion; and n is an integer from 1 to 6. For example, X may be bromide or chloride, or X may be bromide.

In one embodiment, R¹ and R² are hydroxyl protecting groups; R³, R⁴, R⁵, R⁶, R⁷, R⁸, and R⁹ independently, are selected from H; halo; —CN; —NO₂; —Rn; —OH; —OR^{β}; —NH₂; —NHR^{β}; and —N(R^{β})₂; each —R^{β} is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹ is independently selected from a C₃-C₁₄ aryl group; wherein any —R¹ may optionally be substituted with one or more C₁-C₄ alkyl, halo, —OH, —NH₂, —CN, —C≡CH or oxo (═O) groups; X is a counter anion; and n is an integer from 1 to 6. For example, X may be bromide or chloride, or X may be bromide.

In one embodiment, R¹ and R² are hydroxyl protecting groups; R³, R⁴, R⁵, R⁶, R⁷, R⁸, and R⁹ independently, are selected from H; halo; —CN; —NO₂; —R^{β}; —OH; —OR³; and —NH₂; each —R^{β} is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is an integer from 1 to 6. For example, X may be bromide or chloride, or X may be bromide.

In one embodiment, R¹ and R² are hydroxyl protecting groups; R³, R⁴, R⁵, R⁶, R⁷, R⁸, and R⁹ are each H; R¹⁰ is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is an integer from 1 to 4. For example, X may be bromide or chloride, or X may be bromide.

In one embodiment, R¹ and R² are hydroxyl protecting groups; R³, R⁴, R⁵, R⁶, R⁷, R⁸, and R⁹ are each H; R¹⁰ is —[P(Ph)₃]X; X is a counter anion; and n is an integer from 1 to 4. For example, X may be bromide or chloride, or X may be bromide.

In one embodiment, the invention provides a compound of formula (I), wherein: R¹ and R² are independently selected from —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, —OC(C≡CH)H₂, or R¹ and R² together form a C₁₋₄ alkylene group;
R³, R⁴, R⁵, R⁶, R⁷, R⁸, and R⁹, independently, are selected from H; halo; —CN; —NO₂; —R^{β}; —OH; —OR³; —SH; —SR; —SOR^{β}; —SO₂H; —SO₂R^{β}; —SO₂NH₂; —SO₂NHR^{β}; —SO₂N(R⁰)₂; —NH₂; —NHR^{β}; —N(R^{β})₂; —CHO; —COR^{β}; —COOH; —COOR^{β}; and —OCOR^{β}; each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; X is a counter anion;
each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
and
n is an integer from 1 to 14.

In one embodiment, R¹ and R² are independently selected from —C₁₋₄ alkyl, —C(O)R¹³, —C(O)OR¹³, —C(O)NHR¹³, —C(O)N(R¹³)₂, —OCF₃, —OCHF₂, —OC(C≡CH)H₂, or R¹ and R² together form a C₁₋₄ alkylene group; R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; —N(R^{β})₂; —CHO; —COR^{β}; —COOH; —COOR^{β}; and —OCOR^{β}; each —R³ is independently selected from a C₁-C₆ alkyl, C₂-C₆ alkenyl, C₂-C₆ alkynyl or C₃-C₁₄ cyclic group; R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group; X is a counter anion; each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴, each R¹⁴ is independently selected from a halo, —NO₂, —CN, —OH, —NH₂, mercapto, formyl, carboxy, or carbamoyl group; and n is an integer from 1 to 6. For example, X may be bromide or chloride, or X may be bromide.

In one embodiment, R¹ and R² are independently selected from —C₁₋₄ alkyl, —C(O)R¹³, —C(O)OR¹³, —C(O)NHR¹³, —C(O)N(R¹³)₂, —OCF₃, —OCHF₂, —OC(C≡CH)H₂, or R¹ and R² together form a C₁₋₄ alkylene group; R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; and —N(RD)₂; each —R is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹¹ is independently selected from H, or C₁-C₆ alkyl, or C₃-C₁₄ aryl group; X is a counter anion; each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴, each —R¹⁴ is independently selected from methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl; and n is an integer from 1 to 6. For example, X may be bromide or chloride, or X may be bromide.

In one embodiment, R¹ and R² are independently selected from —C₁₋₄ alkyl, —C(O)R¹³, —C(O)NHR¹³, —C(O)N(R¹³)₂, or R¹ and R² together form a C₁₋₄ alkylene group; R³, R⁴, R⁵, R⁶, R⁷, R⁸, and R⁹ independently, are selected from H; halo; —CN; —NO₂; —R^{β}; —OH; —OR^{β}; —NH₂; —NHR^{β}; and —N(R^{β})₂; each —R^{β} is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹¹ is independently selected from a C₃-C₁₄ aryl group; wherein any —R¹ may optionally be substituted with one or more C₁-C₄ alkyl, halo, —OH, —NH₂, —CN, —C≡CH or oxo (═O) groups; X is a counter anion; each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl; and n is an integer from 1 to 6. For example, X may be bromide or chloride, or X may be bromide.

In one embodiment, R¹ and R² are independently selected from —C₁₋₄ alkyl, —C(O)R¹³, —C(O)NHR¹³, —C(O)N(R¹³)₂, or R¹ and R² together form a C₁₋₄ alkylene group; R³, R⁴, R⁵, R⁶, R⁷, R⁸, and R⁹ independently, are selected from H; halo; —CN; —NO₂; —R^{β}; —OH; —OR^{β}; and —NH₂; each —R^{β} is independently selected from a C₁-C₆ alkyl, C₂-C₆ alkenyl, C₂-C₆ alkynyl or C₃-C₁₄ cyclic group; R¹⁰ is —[P(R¹¹)₃]X, wherein each —R¹¹ is a phenyl group; each phenyl group optionally be substituted with one or more C₁-C₄ alkyl, halo, —OH, —NH₂, —CN, —C≡CH or oxo (═O) groups; X is a counter anion; each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl; and n is an integer from 1 to 6. For example, X may be bromide or chloride, or X may be bromide.

In one embodiment, R¹ and R² are independently selected from —OCH₃, —CO^{t}Bu, —CONHCH₃, —CONHCH₂CH₃ and —CON(CH₃)₂, or R¹ and R² together form a methylene group; R³, R⁴, R⁵, R⁶, R⁷, R⁸, and R⁹ are each H; R¹⁰ is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is an integer from 1 to 4. For example, X may be bromide or chloride, or X may be bromide.

In one embodiment, R¹ and R² are independently selected from —OCH₃, —CO^{t}Bu, —CONHCH₃, —CONHCH₂CH₃ or —CON(CH₃)₂, or R¹ and R² together form a methylene group; R³, R⁴, R⁵, R⁶, R⁷, R⁸, and R⁹ are each H; R¹⁰ is —[P(Ph)₃]X; X is a counter anion; and n is an integer from 1 to 4. For example, X may be bromide or chloride, or X may be bromide.

In one embodiment, R¹ and R² are independently selected from —OCH₃, —CO^{t}Bu, —CONHCH₃, —CONHCH₂CH₃ or —CON(CH₃)₂, or R¹ and R² together form a methylene group; R³, R⁴, R⁵, R⁶, R⁷, R⁸, and R⁹ are each H; R¹⁰ is —[P(Ph)₃]X; X is a counter anion; and n is 4. For example, X may be bromide or chloride, or X may be bromide.

In one embodiment, the compounds include a quaternary phosphonium group or quaternary ammonium group and X is a counter anion. Preferably, the counter anion X may be any pharmaceutically acceptable, non-toxic counter ion. For example, X may be bromide or chloride, or X may be bromide.

The counter anion may optionally be singly, doubly or triply charged. As the quaternary group is singly charged, if the counter anion is triply charged then the stoichiometric ratio of the quaternary group to counter anion will typically be 3:1 and if the counter anion is doubly charged then the stoichiometric ratio of the quaternary group to counter anion will typically be 2:1. If both the quaternary group and the counter anion are singly charged then the stoichiometric ratio of the quaternary group to counter anion will typically be 1:1.

If R¹⁰ includes more than one (for example two) quaternary ammonium groups, R¹⁰ will be doubly charged. If the counter anion is triply charged then the stoichiometric ratio of R¹⁰ to counter anion will typically be 3:2 and if the counter anion is doubly charged then the stoichiometric ratio of R¹⁰ to counter anion will typically be 1:1. If the counter anion is singly charged then the stoichiometric ratio of R¹⁰ to counter anion will typically be 1:3.

In one embodiment, the counter anion will be a singly charged anion. Suitable anions X include but are not limited to halides (for example fluoride, chloride, bromide or iodide) or other inorganic anions (for example nitrate, perchlorate, sulfate, bisulfate, or phosphate) or organic anions (for example propianoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonare, ethanesulfonare, 2-hydroxyethanesulfonate, benzenesulfonate, toluene-p-sulfonate, naphthalene-2-sulfonate, camphorsulfonate, ornithinate, glutamate or aspartate). The counter anion may be fluoride, chloride, bromide or iodide. For example, X may be bromide or chloride, or X may be bromide.

In one aspect of any of the above embodiments, the compound of formula (I) has a molecular weight of from 250 to 2,000 Da. Typically, the compound of formula (I) has a molecular weight of from 300 to 1,000 Da. Typically, the compound of formula (I) has a molecular weight of from 350 to 800 Da. More typically, the compound of formula (I) has a molecular weight of from 500 to 750 Da.

A second aspect of the invention provides a compound selected from the group consisting of:

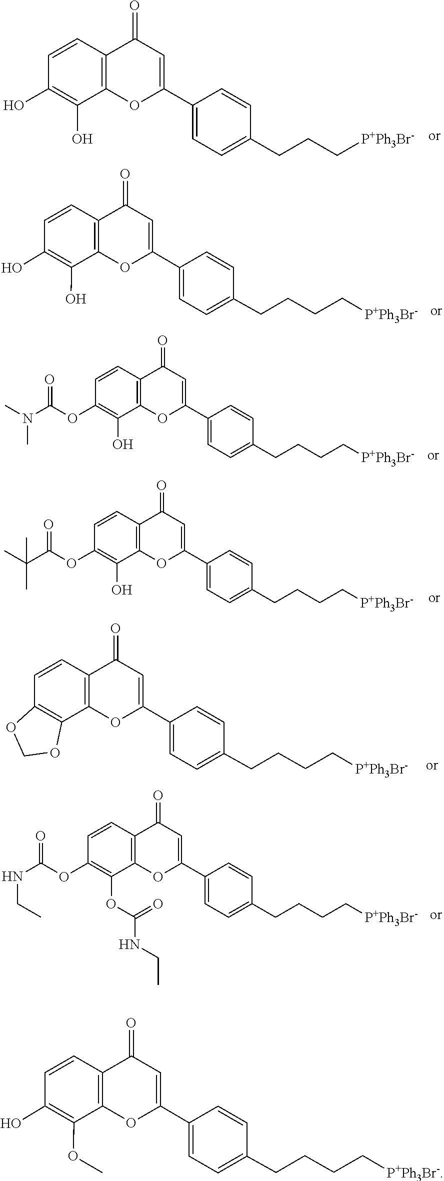

In one embodiment, the compound is selected from:

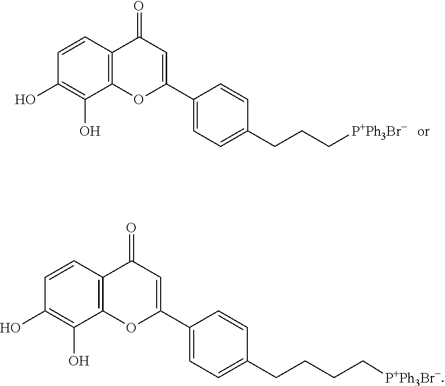

In one embodiment, the compound is selected from:

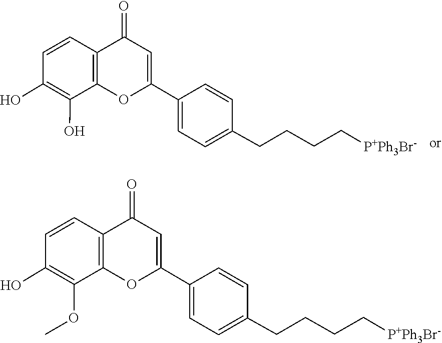

A third aspect of the invention provides a pharmaceutically acceptable multi-salt, solvate or prodrug of any compound of the first or second aspect of the invention.

The compounds of the present invention can be used both in their quaternary salt form (as a single salt). Additionally, the compounds of the present invention may contain one or more (e.g. one or two) acid addition or alkali addition salts to form a multi-salt. A multi-salt includes a quaternary salt group as well as a salt of a different group of the compound of the invention.

For the purposes of this invention, a “multi-salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

The compounds of the present invention can be used both, in quaternary salt form and their multi-salt form. For the purposes of this invention, a “multi-salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt.

Preferably the salt is a mono- or di-lithium, sodium, potassium, magnesium, calcium or ammonium salt. More preferably the salt is a mono- or di-sodium salt or a mono- or di-potassium salt.

Preferably any multi-salt is a pharmaceutically acceptable non-toxic salt. However, in addition to pharmaceutically acceptable multi-salts, other salts are included in the present invention, since they have potential to serve as intermediates in the purification or preparation of other, for example, pharmaceutically acceptable salts, or are useful for identification, characterisation or purification of the free acid or base.

The compounds and/or multi-salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound.

Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses multi-salts and solvates of such prodrugs as described above.

The compounds, multi-salts, solvates and prodrugs of the present invention may contain at least one chiral centre. The compounds, multi-salts, solvates and prodrugs may therefore exist in at least two isomeric forms. The present invention encompasses racemic mixtures of the compounds, multi-salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, multi-salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, multi-salts, solvates and prodrugs of the present invention may be in any polymorphic or amorphous form.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. Typically the use comprises the administration of the compound, multi-salt, solvate, prodrug or pharmaceutical composition to a subject.

An sixth aspect of the invention provides the use of a compound of the first or second aspect, a pharmaceutically effective multi-salt, solvate or prodrug of the third aspect, or a pharmaceutical composition according to the fourth aspect in the manufacture of a medicament for the treatment or prevention of a disease, disorder or condition. Typically the treatment or prevention comprises the administration of the compound, multi-salt, solvate, prodrug or pharmaceutical composition to a subject.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, multi-salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, beta-amyloid 42, tau and phosphor-tau.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, or seventh aspect of the present invention, the disease, disorder or condition is a disease, disorder or condition associated with neurotrophic factors pathways. For example, the disease, disorder or condition may be associated with BDNF pathways

In one embodiment of the fifth, sixth, or seventh aspect of the present invention, the disease, disorder or condition is a mitochondrial disease, disorder or condition. For example, mitochondrial diseases are a group of disorders caused by dysfunctional mitochondria. Dysfunctional mitochondria may exhibit one of the following: impaired Ca influx, energy supply, and/or control of apoptosis. Dysfunctional mitochondria may also or alternatively exhibit increased ROS production.

In one embodiment of the fifth, sixth, or seventh aspect of the present invention, the disease, disorder or condition is related to oxidative stress and/or mitochondrial DNA mutation.

In one embodiment of the fifth, sixth, or seventh aspect of the present invention, the disease, disorder or condition is selected from but not limited to:

(i) central nervous system diseases such as Parkinson's disease, Alzheimer's disease, dementia, motor neuron disease, Huntington's disease, cerebral malaria, and brain injury from pneumococcal meningitis;
(ii) depression, anxiety, amytrophic later sclerosis, Autism spectrum disorders, Rett syndrome, epilepsy, Parkinson's disease, post-traumatic stress disorder, diabetic neuropathy, peripheral neuropathy, obesity, or stroke;
(iii) neurological disorders, neuropsychiatric disorders, and metabolic disorders.

Examples of neurological and neuropsychiatric disorders include depression, anxiety, Alzheimer's, CNS injuries, and the like. Examples of metabolic disorders include obesity and hyperphagia;

(iv) mental disorders and conditions include, but are not limited to, acute stress disorder, adjustment disorder, adolescent antisocial behaviour, adult antisocial behaviour, age-related cognitive decline, agoraphobia, alcohol-related disorder, Alzheimer's, amnestic disorder, anorexia nervosa, anxiety, attention deficit disorder, attention deficit hyperactivity disorder, autophagia, bereavement, bibliomania, binge eating disorder, bipolar disorder, body dysmorphic disorder, bulimia nervosa, circadian rhythm sleep disorder, cocaine-addition, dysthymia, exhibitionism, gender identity disorder, Huntington's disease, hypochondria, multiple personality disorder, obsessive-compulsive disorder (OCD), obsessive-compulsive personality disorder (OCPD), posttraumatic stress disorder (PTSD), Rett syndrome, sadomasochism, and stuttering;
(v) cyclothymic disorders with compounds disclosed herein;
(vi) amyotrophic lateral sclerosis (ALS) or a central nervous system injury. A central nervous system injury includes, for example, a brain injury, a spinal cord injury, or a cerebrovascular event (e.g., a stroke);
(vii) cardiovascular diseases, such as coronary artery disease, heart attack, abnormal heart rhythms or arrhythmias, pericardial disease, heart failure, heart valve disease, congenital heart disease, heart muscle disease (cardiomyopathy), aorta disease and vascular disease;
(viii) ageing related diseases and/or ageing per se; and
(ix) the subject in need thereof can be a patient diagnosed as suffering from being overweight or obese.

Anxiety can be a symptom of an underlying health issue such as chronic obstructive pulmonary disease (COPD), heart failure, or heart arrhythmia.

In one embodiment, the disease, disorder or condition is a central nervous system disease or a cardiovascular disease.

In one embodiment, the compounds may be used for treating or preventing a neurodegenerative disorder. For example, the compounds may be used for treating or preventing Alzheimer's Disease, Parkinson's Disease, or ischemia.

In one embodiment, the compounds may be used for treating or preventing rare CNS disorders. For example, the compounds may be used to treat or prevent Rett Syndrome, or KBG Syndrome.

In one embodiment, the compounds may be used for treating or preventing anti-aging or mitochondria linked disorders.

In one embodiment, the disease, disorder or condition is selected from but not limited to Parkinson's disease, Alzheimer's disease, and depression.

In one embodiment, the disease, disorder or condition is Alzheimer's disease.

An eighth aspect of the invention provides a method of modulating neurotrophic factors pathways (such as BDNF pathways), the method comprising the use of a compound of the first or second aspect of the invention, or a pharmaceutically acceptable multi-salt, solvate or prodrug of the third aspect of the invention, or a pharmaceutical composition of the fourth aspect of the invention, to modulate neurotrophic factors pathways (such as BDNF pathways).

A ninth aspect of the invention provides a method of modulating mitochondrial function, the method comprising the use of compound of the first or second aspect of the invention, or a pharmaceutically acceptable multi-salt, solvate or prodrug of the third aspect of the invention, or a pharmaceutical composition of the fourth aspect of the invention, to modulate mitochondrial function.

In one embodiment of the ninth aspect of the present invention, modulating mitochondrial function includes: modulating Ca influx, energy supply, control of apoptosis and/or ROS production.

In one embodiment of the ninth aspect of the present invention, the method comprises delivering a compound of the first or second aspect of the invention to the mitochondria of a cell.

In one embodiment of the eighth or ninth aspect of the present invention, the method is performed ex vivo or in vitro, for example in order to analyse the effect on cells of neurotrophic factors pathways modulation or mitochondrial function modulation.

In another embodiment of the eighth or ninth aspect of the present invention, the method is performed in vivo. For example, the method may comprise the step of administering an effective amount of a compound of the first or second aspect, or a pharmaceutically acceptable multi-salt, solvate or prodrug of the third aspect, or a pharmaceutical composition of the fourth aspect, to thereby modulate neurotrophic factors pathways or modulate mitochondrial function. Typically the administration is to a subject in need thereof.

Alternately, the method of the eighth or ninth aspect of the invention may be a method of modulating factors pathways or modulating mitochondrial function in a non-human animal subject, the method comprising the steps of administering the compound, multi-salt, solvate, prodrug or pharmaceutical composition to the non-human animal subject and optionally subsequently mutilating or sacrificing the non-human animal subject. Typically such a method further comprises the step of analysing one or more tissue or fluid samples from the optionally mutilated or sacrificed non-human animal subject.

Unless stated otherwise, in any aspect of the invention, the subject may be any human or other animal. Typically, the subject is a mammal, more typically a human or a domesticated mammal such as a cow, pig, lamb, goat, horse, cat, dog, etc. Most typically, the subject is a human.

Any of the medicaments employed in the present invention can be administered by oral, parental (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder or disease to be treated or prevented.

For oral administration, the compounds, multi-salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, multi-salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For transdermal and other topical administration, the compounds, multi-salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, multi-salts, solvates or prodrugs of the present invention will, of course, vary with the disorder or disease to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

### Examples—Compound Synthesis

Compounds of the invention are synthesised employing a route of synthesis shown below. The general route of synthesis is illustrated below by reference to the synthesis of a specific compound. However, this is merely illustrative of a more general synthesis that can be employed to synthesise all compounds of the invention.

### Route of Synthesis:

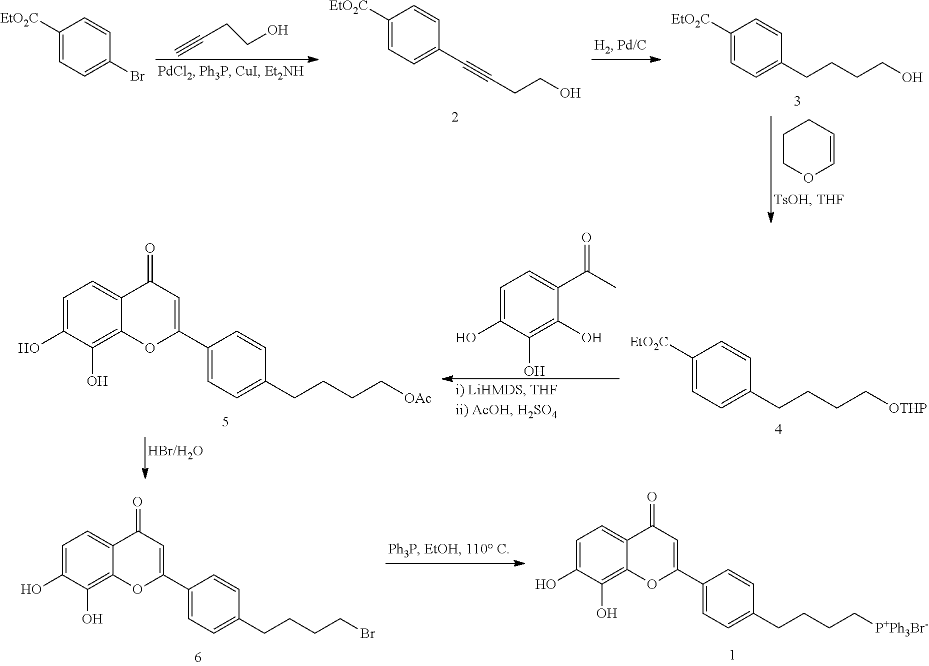

### Examples—Compound Synthesis

All solvents, reagents and compounds were purchased and used without further purification unless stated otherwise.

Abbreviations

LiHMDS—Lithium bis(trimethylsilyl)amide

### THF—Tetrahydrofuran

### THP—Tetrahydropyran

Pd/C—Palladium on carbon (10 wt. % loading)
AcOH—Acetic acid

### DCM—Dichloromethane

### MeOH—Methanol

### EtOH—Ethanol

### Et₂NH—Diethylamine

TsOH—Toluenesulfonic acid

Synthesis of Compound A/SND118

### Ethyl 4-(4-hydroxybut-1-ynyl)benzoate (2)

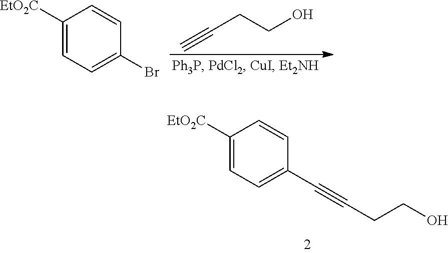

This Sonogashira coupling following a published procedure [Radeke H et al, 2007] provided 82% yield of 2.

A suspension of ethyl 4-bromobenzoate (50 g, 0.218 mol) in diethylamine (700 mL) was stirred at room temperature under nitrogen and treated with PdCl₂ (1.93 g) and triphenylphosphine (0.57 g). The mixture was de-gassed by bubbling nitrogen through for 30 min. CuI (0.42 g) and 3-butyn-1-ol (15.3 g, 0.218 mol) were added and the mixture continued at room temperature.

After 20 hours more PdCl₂ (0.2 g), triphenylphosphine (0.06 g) and 3-butyn-1-ol (1.5 g) were added and continued at room temperature.

After 44 hours the reaction mixture was evaporated in vacuo. Column chromatography of the residue provided ethyl 4-(4-hydroxybut-1-ynyl)benzoate (2) as a waxy solid, 39.3 g, 82-5%.

1H NMR (300 MHz, CDCl₃): δ 8.00 ppm (d, 2H), 7.48 (d, 2H), 4.39 (q, 2H), 3.84 (t, 2H), 2.72 (t, 2H), 2.88 (br s, 1H), 1.40 (t, 3H).

### Ethyl 4-(4-hydroxybutyl)benzoate (3)

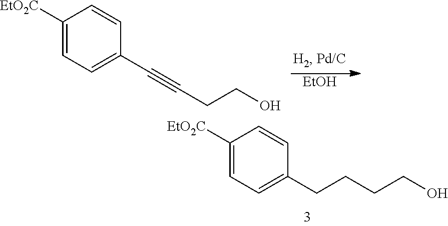

Hydrogenation at 40 psi pressure of hydrogen provided the saturated product (3) A solution of ethyl 4-(4-hydroxybut-1-ynyl)benzoate (41.5 g, 0.179 mol) in EtOH (300 mL) was treated with 10% Pd/C (9.51 g) and hydrogenated at 40 psi at room temperature. After 18 hours the catalyst was removed by filtration and the filtrate was evaporated in vacuo to provide ethyl 4-(4-hydroxybutyl)benzoate as an amber oil, 37.27 g, 93.8%.

1H NMR (300 MHz, CDCl₃): δ 7.98 ppm (d, 2H), 7.26 (d, 2H), 4.38 (q, 2H), 3.65 (t, 2H), 2.70 (t, 2H), 1.45-1.80 (m, 4H), 1.55 (br s, 1H), 1.40 (t, 3H).

### Ethyl 4-(4-tetrahydropyran-2-yloxybutyl)benzoate (4)

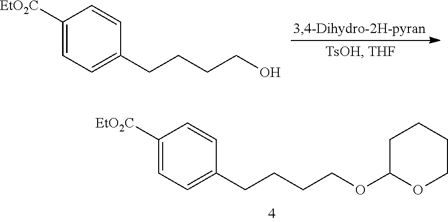 [SMILES: CCOC(=O)c1ccc(CCCCO)cc1 ; CCOC(=O)c1ccc(CCCCOC2CCCCO2)cc1]

3,4-Dihydropyran (16.4 g, 0.195 mol) in THF (50 mL) was added dropwise to a stirred solution of ethyl 4-(4-hydroxybutyl)benzoate (31.0 g, 0.139 mol) containing p-toluenesulphonic acid monohydrate (1.33 g, 6.97 mmol) in THF (320 mL) at 0° C.

Warmed to room temperature for 18 hours then the reaction mixture was added to sat NaHCO₃ (700 ml) and extracted with diethyl ether (2×500 mL). The combined extracts was washed with sat. brine, dried (MgSO₄) and evaporated in vacuo.

Ethyl 4-(4-tetrahydropyran-2-yloxybutyl)benzoate was obtained with good purity as an amber oil, 44.64 g, 99.8%

1H NMR (300 MHz, CDCl₃): δ 7.87 ppm (d, 2H), 7.17 (d, 2H), 4.48 (t, 1H), 4.28 (q, 2H), 3.60-3.85 (m, 3H), 3.25-3.45 (m, 2H), 2.60 (t, 2H), 1.35-1.8 (m, 9H), 1.28 (t, 3H)

### 4-[4-(7,8-dihydroxy-4-oxo-chromen-2-yl)phenyl]butyl acetate (5)

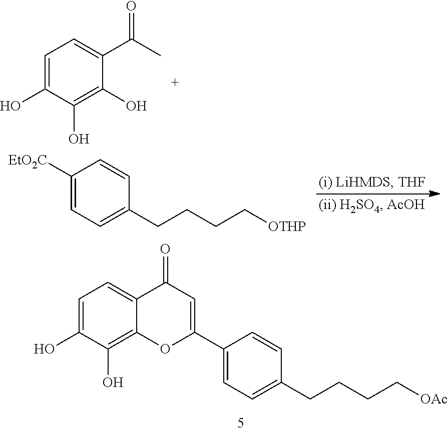

This flavone formation was carried out in two stages. The initial condensation was followed by treatment of the resulting diketone intermediate with acetic acid containing a small amount of sulphuric acid at 100° C. These conditions, in addition to effecting cyclisation to the flavone also removed the THP protection providing the acetate.

1M LiHMDS/THF solution (98.1 mL, 98.1 mmol) was added dropwise, over 30 min to a stirred solution of 2,3,4-trihydroxyacetophenone (3.39, 19.9 mmol) in THF (170 mL) at −70° C. Stirred 1 hour at −70° C. then warmed to −10° C. for 1 hour. Cooled back to −70° C. and a solution of ethyl 4-(4-tetrahydropyran-2-yloxybutyl)benzoate (6-3 g, 19.6 mmol) in THF (30 mL) was added dropwise over 20 min. The reaction mixture was continued at −70° C. for 1 hour then warmed to room temperature.

After 18 hours the reaction mixture was poured into ice-water (1 L) and acidified by addition of 2N HCl. Extracted with EtOAc (3×300 mL) and the combined extracts was washed with saturated brine (300 mL), dried (MgSO₄) and evaporated in vacuo. Brown oil, 11.72 g.

This oil was dissolved in glacial acetic acid (68 mL) and conc. H₂SO₄ (0.3 mL) was added. Stirred under nitrogen and heated to 100° C. for 1 hour. The dark solution was cooled, poured onto ice-water (330 mL) and extracted with EtOAc (3×150 mL). The combined extracts was washed with saturated brine (4×150 mL) and dried (MgSO₄).

Evaporated in vacuo to leave a dark oil/solid. This was triturated with dichloromethane (DCM) (30 mL) then petroleum ether (7-5 mL) was added. Stirred and cooled in an ice bath then the solid was filtered off, washed with DCM/petrol (4:1) then with petrol. 4-[4-(7,8-Dihydroxy-4-oxo-chromen-2-yl)phenyl]butyl acetate was obtained as a brown solid, 4.64 g, 64.2%.

1H NMR (300 MHz, d6-DMSO): δ 10.30 ppm (br s, 1H), 9.44 (br s, 1H), 8.07 (d, 2H), 7.40 (d, 2H), 7.40 (d, 1H), 6.95 (d, 1H), 6.83 (s, 1H), 4.02 (t, 2H), 2.70 (t, 2H), 2.00 (s, 3H), 1.50-1.70 (m, 4H).

### 2-T4-(4-bromobutyl)phenyl-7,8-dihydroxychromen-4-one (6)

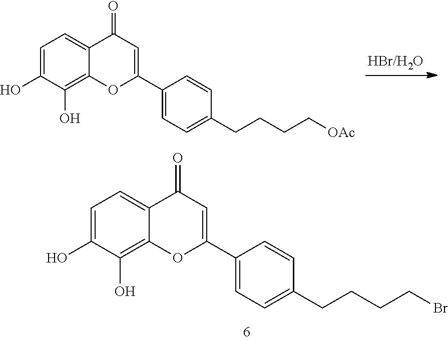

A suspension of 4-[4-(7,8-dihydroxy-4-oxo-chromen-2-yl)phenyl]butyl acetate (4.60 g, 12.5 mmol) in 62% aqueous HBr (10.9 mL, 125 mmol) was stirred and heated to 80° C. After 5 hours the reaction mixture (light brown suspension) was cooled and treated with EtOAc (150 mL) and water (50 mL). The aqueous phase was extracted with EtOAc (2×30 mL). The combined organics was washed with water (2×100 mL), dried (MgSO₄) and evaporated in vacuo to leave a brown solid/oil, 4.58 g.

Purification by column chromatography (DCM/MeOH, 96:4) provided 2-[4-(4-bromobutyl)phenyl]-7,8-dihydroxychromen-4-one as a yellow solid, 2.139, 44%.

1H NMR (300 MHz, d6-DMSO): δ 10.30 ppm (br s, 1H), 9.44 (br s, 1H), 8.07 (d, 2H), 7.41 (d, 2H), 7.40 (d, 1H), 6.95 (d, 1H), 6.83 (s, 1H), 3.58 (t, 2H), 2.70 (t, 2H), 1.65-1.88 (m, 4H).

### 4-[4-(7,8-dihydroxy-4-oxo-chromen-2-yl)phenyl]butyltriphenylphosphonium bromide (1) (Compound A)

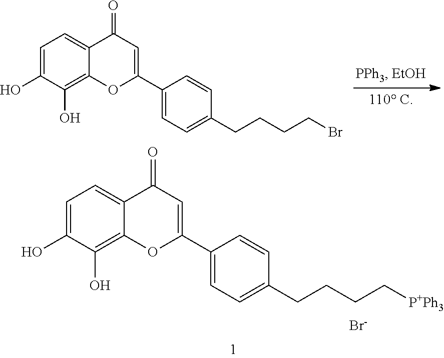

This reaction involved heating to 110° C. in a sealed vessel and was not a particularly clean reaction so required column chromatography for purification. Solvent removal from the isolated product proved difficult. Material from two separate batches was combined in ethanol solution and evaporated to a solid.

A solution of 2-[4-(4-bromobutyl)phenyl]-7,8-dihydroxychromen-4-one (1.80 g, 4.62 mmol) in EtOH (70 mL) was treated with triphenylphosphine (1.58 g, 6.01 mmol) and stirred in a sealed glass tube while heated to 110° C. After 66 hours the solution was cooled and evaporated to a yellow foam, 3.35 g.

Column chromatography (DCM/MeOH. 95:5 gradient to 90:10) provided the product at 93% purity. Further column chromatography of this material (DCM/MeOH (93:7) improved the purity to >95%, providing 4-[4-(7,8-dihydroxy-4-oxo-chromen-2-yl)phenyl]butyl-triphenylphosphonium bromide as a yellow foam, 0.75 g, 25% yield. This was combined with a second batch of similar purity prepared by the same procedure. The combined material was evaporated from ethanol to a yellow foam. After crushing to a powder and drying under vacuum 4-[4-(7,8-dihydroxy-4-oxo-chromen-2-yl)phenyl]butyl-triphenylphosphonium bromide was obtained as a yellow solid, 1.364 g, 21% yield with 97.3% HPLC purity.

1H NMR (300 MHz, d6-DMSO): δ 10.35 ppm (br s, 1H), 9.50 (br s, 1H), 8.02 (d, 2H), 7.7-7.95 (m, 15H), 7.40 (d, 1H), 7.35 (d, 2H), 6.96 (d, 1H), 6.84 (s, 1H), 3.65 (m, 2H), 2.70 (t, 2H), 1.80 (m, 2H), 1.48-1.65 (m, 2H).

Compound A is also referred to as compound SND118.

Other compounds can be synthesised in essentially the same way. Some further examples are provided below.

### Synthesis of SND127—4-[4-[7-(Isopropylcarbamoyloxy)-8-hydroxy-4-oxo-chromen-2-yl]phenyl]butyltriphenylphosphonium bromide

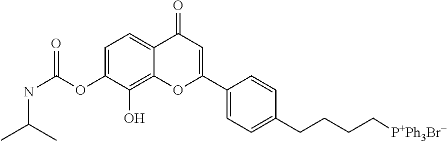

Compound was synthesised using the following general scheme:

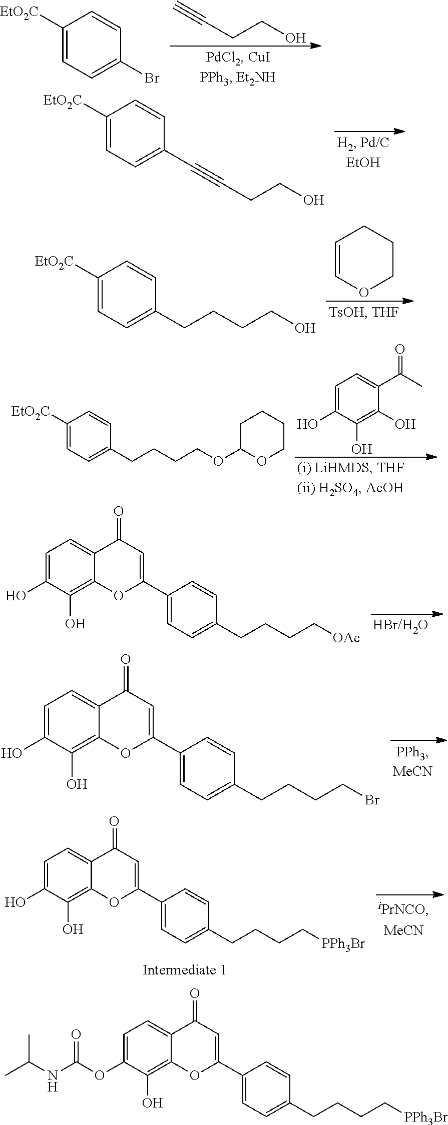

Initially it was attempted to limit the formation of the carbamates to the monocarbamates using base as the biscarbamate compounds appeared to be particularly sensitive to base. However attempted to use either K₂CO₃ or DBU mostly gave starting material by LCMS. The next attempt was to use a slight excess of isopropyl isocyanate (1.2 equivalents) to form the monocarbamate product. Upon cooling, a solid formed, which was filtered and further purified by chromatography (DCM/MeOH) to give the target.

This compound was analysed using SFC conditions, similar to the other compounds in this series, and showed a purity of 99%. The amount of compound obtained was 0.41 g, with a yield of 36% from intermediate 1.

### Experimental Procedure:

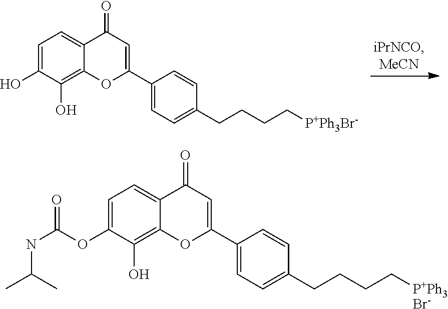

In two vials, a solution of 4-[4-(7,8-dihydroxy-4-oxo-chromen-2-yl) phenyl]butyltriphenylphosphonium bromide (0.5 g, 0.75 mmol) in MeCN (6 mL) was heated to 50° C., isopropyl isocyanate (0.09 mL, 0.9 mmol) and the mixture stirred for 1 h. The solution was cooled and the solids from the vials filtered off, washing through with a small amount of MeCN. The combined solids were then purified by column chromatography (DCM/MeOH, from 0 to 20%) to give an off-white solid.

¹H NMR (400 MHz, d6-DMSO): δ 10.96 (1H, s, br), 8.04 (1H, d, J=7.8 Hz), 7.92-7.86 (5H, m), 7.83-7.72 (13H, m), 7.32 (2H, d, J=8.3 Hz), 7.06 (1H, d, J=8.8 Hz), 6.93 (1H, s), 3.75-3.56 (3H, m), 2.72 (2H, t J=7.4 Hz), 1.79 (2H, quint. J=7.4 Hz), 1.62-1.49 (2H, m), 1.19 (6H, d, J=6.6 Hz)

### Synthesis of SND124—4-[4-[7,8-Bis(ethylcarbamoyloxy)-4-oxo-chromen-2-yl]phenyl]-butyltriphenylphosphonium bromide

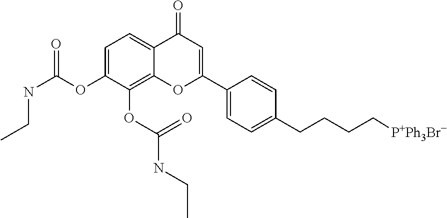

Compound SND124 was synthesised using the general scheme below:

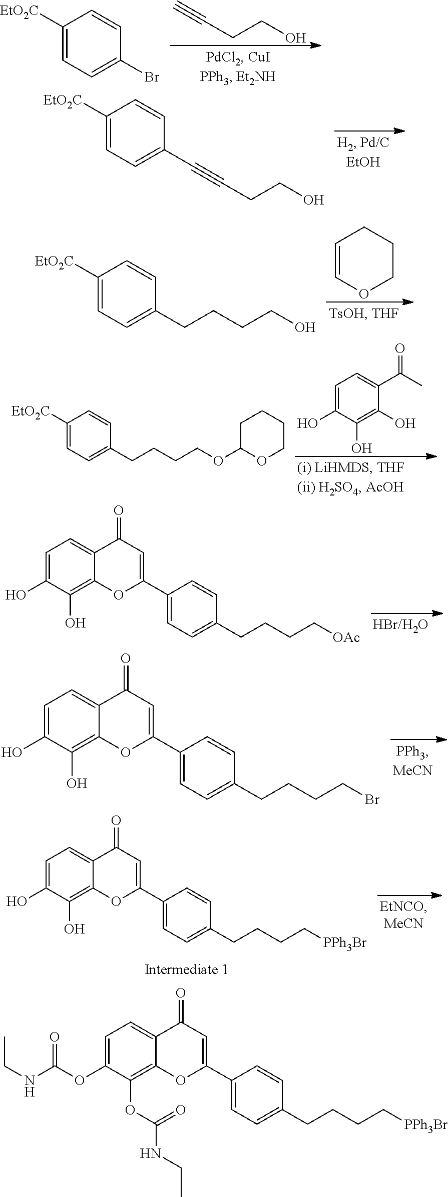

Dissolving the intermediate phosphonium salt in acetonitrile at 50° C. and adding a large excess of ethyl isocyanate gave the desired compound with good conversion by TLC in 1 h. Purification of this material was by chromatography with DCM/MeOH. Ascertaining the purity of the material by standard aqueous HPLC conditions was not possible due to degradation of the material under these conditions but SFC conditions showed a purity of 99%. The amount of compound obtained was 1.61 g, with a yield of 73% from intermediate 1.

### Experimental Procedure:

Ethyl isocyanate (2.4 mL, 31 mmol) was added to a solution of 4-[4-(7,8-dihydroxy-4-oxo-chromen-2-yl)phenyl]butyltriphenylphosphonium bromide (2 g, 3.1 mmol) in MeCN (30 mL) at 50° C. and the mixture stirred for 1 h. The solution was cooled, the solvent removed and the residue was purified by column chromatography (DCM/MeOH, from 0 to 20% MeOH) to give the product as an off-white solid (1.61 g, 73%).

¹H NMR (400 MHz, d6-DMSO): δ 8.33 (1H, t, J=5.5 Hz), 8.08 (1H, t, J=5.5 Hz), 7.93-7.72 (19H), 7.39-7.32 (3H, m), 7.07 (1H, s), 3.69-3.57 (2H, m), 3.23-3.07 (4H, m), 2.73 (2H, t, J=7.6 Hz), 1.85-1.75 (2H, m), 1.61-1.49 (2H, m), 1.17-1.08 (6H, m)

Synthesis of SND 126

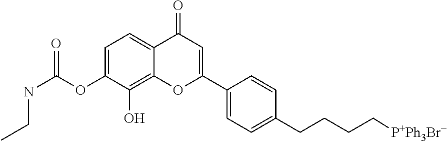

Compound was synthesised using the following scheme:

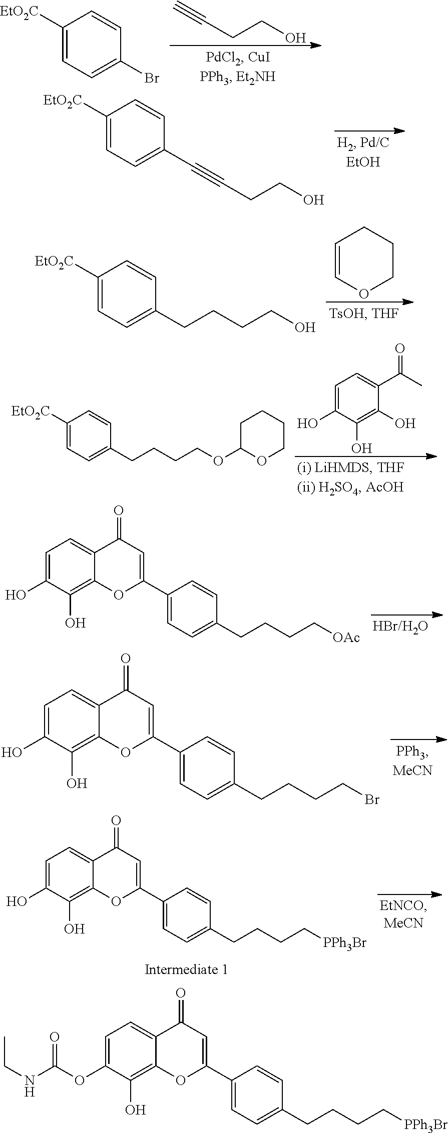

The key intermediate 1 in scheme 1 was used to synthesise the target molecules.

This compound was synthesised using ethyl isocyanate in acetonitrile as the reaction conditions and using a slight excess of ethyl isocyanate (1.2 equivalents) to form the monocarbamate product. Some of the analogous monocarbamate and biscarbamate were formed, so starting from 1 g of the starting material would allow for some room in the chromatography to remove the impurities and achieve the target amount. The material was subjected three times to chromatography (DCM/MeOH) to give the target.

This compound was analysed using SFC conditions, similar to the other compounds in this series, and showed a purity of 99%. The amount obtained was 0.72 g, with a yield of 65% from intermediate 1.

### Experimental Procedure

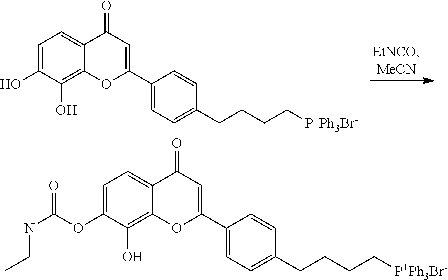

Ethyl isocyanate (0.15 mL, 1.8 mmol) was added to a solution of 4-[4-(7,8-dihydroxy-4-oxochromen-2-yl)phenyl]butyltriphenylphosphonium bromide (1.0 g, 1.5 mmol) in MeCN (20 mL) at 50° C. and the mixture stirred for 1 h. The solution was cooled, concentrated and the residue purified by column chromatography twice (DCM/MeOH, from 0 to 20% MeOH) to give the product as an offwhite solid. A further column using DCM/DCM+10% MeOH, 0 to 100%) gave the product as an offwhite solid.

¹H NMR (400 MH z, d6-DMSO): δ 10.96 (1H, s, br), 8.10 (1H, t, J=5-7 Hz), 7.93-7.84 (5H, m), 7.84-7.71 (13H, m), 7.34 (2H, d, J=8.3 Hz), 7.06 (1H, d, J=8.8 Hz), 6.93 (1H, s), 3.70-3.57 (2H, m), 3.18 (2H, quint., J=6.0 Hz), 2.73 (2H, t J=7.4 Hz), 1.80 (2H, quint. J=7.2 Hz), 1.61-1.49 (2H, m), 1.15 (3H, t, J=7.2 Hz)

### Synthesis of SND 125—4-[4-[7,8-Bis(isopropylcarbamoyloxy)-4-oxo-chromen-2-yl]phenyl]-butyltriphenylphosphonium bromide

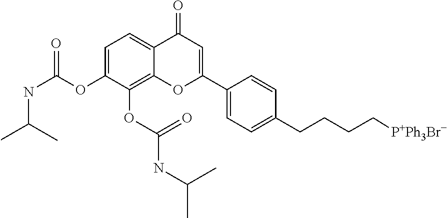

Compound was synthesised using the following scheme:

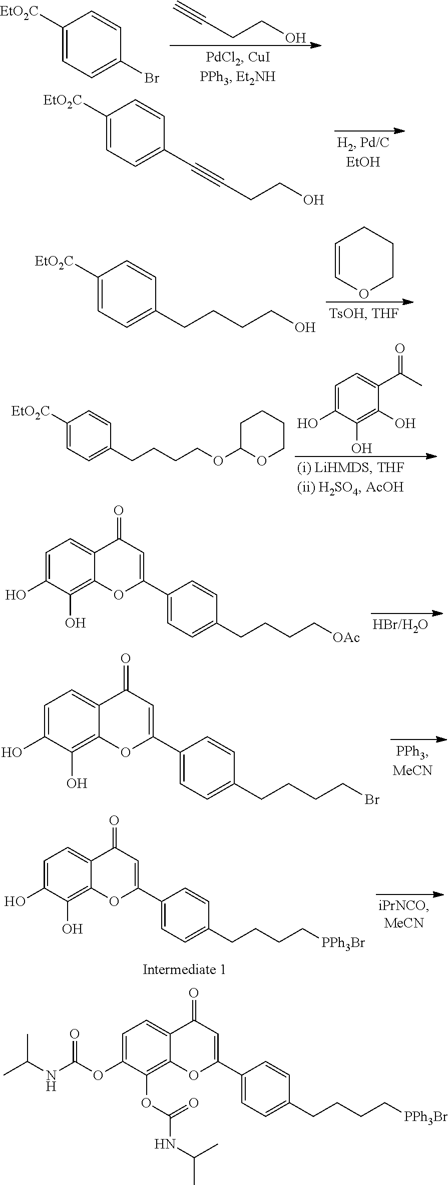

Dissolving the intermediate phosphonium salt in acetonitrile at 50° C. and adding a large excess of ethyl isocyanate gave the desired compound with good conversion by TLC in 1 h. Purification of this material was by chromatography with DCM/MeOH twice. After a period of storage, the material did appear to have slightly degraded and was re-purified for a third time to get the purity level up to the required standard. SFC conditions showed a purity of 99%. The amount obtained was 0.1 g, with an yield of 16% from intermediate 1, with the most likely reason for the poor yield being due to the repeated chromatography to reach the desired purity level.

### Experimental Procedure:

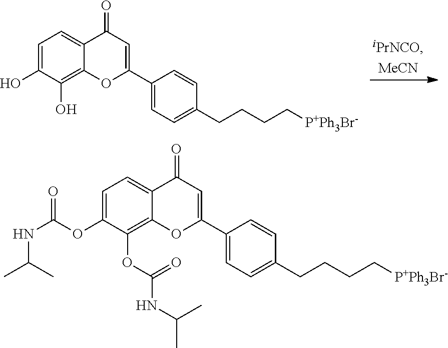

In two vials, a solution of 4-[4-(7,8-dihydroxy-4-oxo-chromen-2-yl)phenyl]butyltriphenyl-phosphonium bromide (0.25 g, 0.39 mmol) in MeCN (4 mL) was heated to 50° C. and isopropyl isocyanate (0.38 mL) was added. The mixtures were stirred for 1 h, at which point the starting material was consumed by TLC. The solutions were cooled, combined, the solvent removed and the residue was purified by column chromatography (DCM/MeOH, from 0 to 20% MeOH) three times to give the product as an off-white solid (0.1 g, 16%).

¹H NMR (400 MHz, d6-DMSO): δ 8.25 (1H, d, J=7.7 Hz), 8.04 (1H, d, J=7.7 Hz), 7.95-7.85 (6H, m), 7.84-7.70 (12H, m), 7.35-7.30 (3H, m), 7.08 (1H, s), 3.74-3.56 (4H, m), 2.73 (2H, t, J=7.2 Hz), 1.80 (2H, quint., J=7.1 Hz), 1.56 (2H, m), 1.21-1.12 (12H, m)

### Synthesis of SND135—(4-(4-(7-hydroxy-8-methoxy-4-oxo-4H-chromen-2-yl)phenyl)butyl)triphenylphosphonium bromide

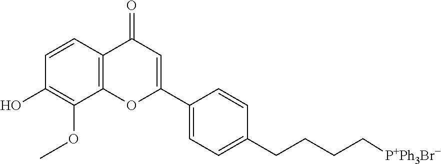

Compound was synthesised using the scheme below:

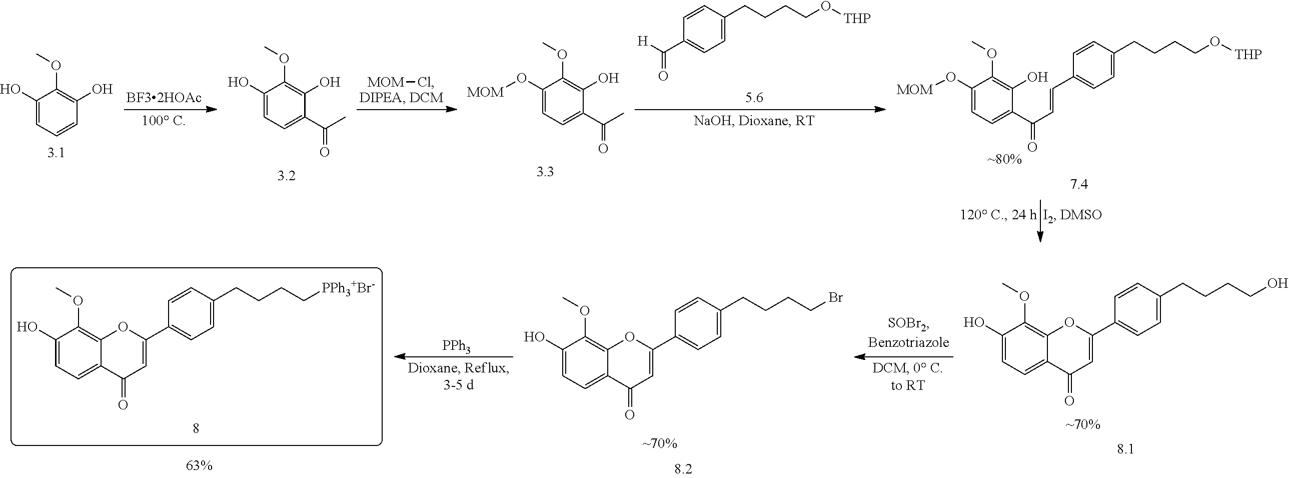

### 1-(2,4-dihydroxy-3-methoxyphenyl)ethan-1-one (3.2)

The solution of 2-methoxybenzene-1,3-diol (3.1) (0.501 g, 1.00 Eq, 3.57 mmol) in boron trifluoride-acetic acid complex (ca. 33% BF₃, 3.36 g, 2.48 mL, 5.00 Eq, 17.9 mmol) was heated to 100° C. for 180 min. The mixture was then poured into water and extracted with 20 mL DCM (3×) (Note: a leak occurred during the workup, so part of the product was lost and the yield cannot be final). The combined organic extracts were washed with brine, dried over anhydrous Na₂SO₄ and concentrated in vacuo. Resulting product 1-(2,4-dihydroxy-3-methoxyphenyl)ethan-1-one (3.2) (0.210 g, 1.15 mmol, 32.2%) was collected as dark yellow crystals.

### (E)-1-(2-hydroxy-3-methoxy-4-(methoxymethoxy)phenyl)-3-(4-(4-((tetrahydro-2H-pyran-2-yl)oxy)butyl)phenyl)prop-2-en-1-one (7.4)

To a solution of 1-(2-hydroxy-3-methoxy-4-(methoxymethoxy)phenyl)ethan-1-one (3-3) (5.00 g, 1 Eq, 22.1 mmol) and 4-(4-((tetrahydro-2H-pyran-2-yl)oxy)butyl)benzaldehyde (5.6) (6.96 g, 1.2 Eq, 26.5 mmol) in dioxane (100 mL) was added, at room temperature, aqueous sodium hydroxide (97.2 g, 97.2 mL, 50% Wt, 55 Eq, 1.22 mol). The reaction was stirred for 24 h at room temperature and controlled with LCMS until maximum conversion was reached. The solution was neutralized using citric acid, and extracted with EtOAc. The organic layers were combined, washed with brine, dried over Na₂SO₄ and concentrated in vacuo. Obtained crude material was purified by column chromatography, yielding (E)-1-(2-hydroxy-3-methoxy-4-(methoxymethoxy)phenyl)-3-(4-(4-((tetrahydro-2H-pyran-2-yl)oxy)butyl)phenyl)prop-2-en-1-one (7.4) (8.67 g, 16 mmol, 72%, 86% Purity) as a dark-orange thick oil.

### 7-hydroxy-2-(4-(4-hydroxybutyl)phenyl)-8-methoxy-4H-chromen-4-one (8.1)

A stirred solution of (E)-1-(2-hydroxy-3-methoxy-4-(methoxymethoxy)phenyl)-3-(4-(4-((tetrahydro-2H-pyran-2-yl)oxy)butyl)phenyl)prop-2-en-1-one (7.4) (6.000 g, 1 Eq, 12.75 mmol) and iodine (323.6 mg, 0.1 Eq, 1.275 mmol) in DMSO (100 mL) was heated to 120° C. for 48 hours. Upon LCMS-confirmed completion, the mixture was cooled and poured into cold water. The mixture was extracted with ethyl acetate (4×200 mL). The combined organic phase was washed with saturated sodium thiosulfate, water and brine successively. Then the organic layer was dried with anhydrous Na₂SO₄ and concentrated in vacuo. 7-hydroxy-2-(4-(4-hydroxybutyl)phenyl)-8-methoxy-4H-chromen-4-one (8.1) (3.92 g, 8.8 mmol, 69%, 76% Purity) was obtained as a viscous dark orange oil, which solidifies upon applying friction.

### 2-(4-(4-bromobutyl)phenyl)-7-hydroxy-8-methoxy-4H-chromen-4-one (8.2)

To a solution of the 7-hydroxy-2-(4-(4-hydroxybutyl)phenyl)-8-methoxy-4H-chromen-4-one (8.1) (1.50 g, 1.0 Eq, 4.41 mmol) in DCM at 0° C. was added 1H-benzo[d][1,2,3]triazole (682 mg, 1.30 Eq, 5.73 mmol) and a drop of DMF (32.2 mg, 0.1 Eq, 441 μmol), followed by sulfurous dibromide (1.19 g, 444 μL, 1.30 Eq, 5.73 mmol). The mixture was allowed to warm to room temperature and then the reaction progress was monitored by LCMS. Upon completion, the mixture was quenched with saturated aqueous NaHCO₃, and extracted with DCM (3×100 mL). The combined organic layers were washed with brine, dried (Na₂SO₄), and concentrated in vacuo. The resulting oil was purified by column chromatography (SiO₂, 0-20% MeOH/DCM) to provide 2-(4-(4-bromobutyl)phenyl)-7-hydroxy-8-methoxy-4H-chromen-4-one (8.2) (0.938 g, 2.33 mmol, 52.8%) as a light-brown solid.

### (4-(4-(7-hydroxy-8-methoxy-4-oxo-4H-chromen-2-yl)phenyl)butyl)triphenylphosphonium bromide (8)

To a solution of 2-(4-(4-bromobutyl)phenyl)-7-hydroxy-8-methoxy-4H-chromen-4-one (8.2) (0.352 g, 1.0 Eq, 873 μmol) and sodium iodide (19.6 mg, 0.15 Eq, 131 μmol) in dioxane (15 mL) was added triphenylphosphine (6.87 g, 30 Eq, 26.2 mmol) and the resulting mixture was heated to reflux (105° C.). Reaction progress was controlled by TLC (DMC/MeOH—9:1). Upon completion, which took 18 hours, the solvent was removed in vacuo and the residue was combined with a previous batch (#53, 250 mg) and triturated with water/toluene/acetone. Part of the solid remained undissolved in DCM and appeared to be the product (batch A, 425 mg, yellow powder, 97% purity). The DCM filtrate was purified by column chromatography (SiO₂, 0-20% MeOH/DCM). yielding (4-(4-(7-hydroxy-8-methoxy-4-oxo-4H-chromen-2-yl)phenyl)butyl)triphenylphosphonium bromide (8), (batch B, 115 mg, brown-yellow powder, 97% purity). Combined, 52% yield.

### Examples—Biological Studies

### Compounds

The following nomenclature is used to refer to the following compounds.

| Compound | Structure |
|---|---|
| SND118 | 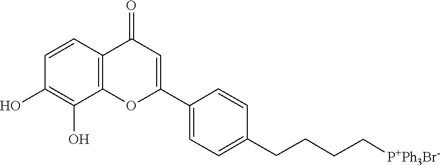 |
| SND121 | 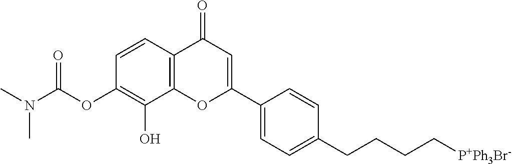 |
| SND122 | 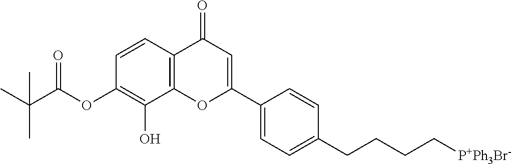 |
| SND123 | 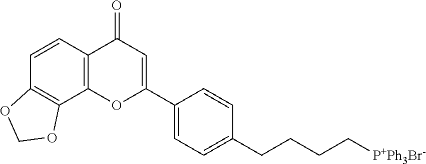 |
| SND124 | 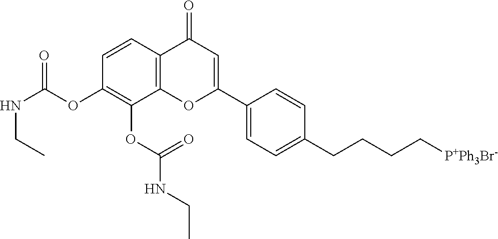 |
| SND135 | 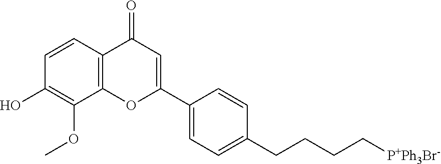 |

### General Methods

### Cell Culture

For neuronal cultures, primary cultures of cortical neurons were prepared from embryonic day 17 (E17) OF₁ mice embryos (Charles River Laboratories) as previously described [Allaman I., Pellerin L., Magistretti P. J. (2004) Glucocorticoids modulate neurotransmitter-induced glycogen metabolism in cultured cortical astrocytes. J. Neurochem. 88, 900-908] or from C57BL/6JRccHsd mice at E18. Animals were sacrificed and embryos were dissected in Calcium and Magnesium free Hanks Balanced Salt Solution (CMF-HBSS) containing 15 mM HEPES and 10 mM NaHCO₃, pH 7.2. Embryos were decapitated, skin and skull gently removed and hemispheres were separated. After removing meninges and brain stem, the hippocampi and cortices were isolated, chopped with a sterile razor blade in Chop solution (Hibernate-E without Calcium containing 2% B-27) and digested in 2 mg/ml papain (Worthington) dissolved in Hibernate-E without Calcium for 30 minutes (±5 min) at 30° C. Cortices were triturated for 10-15 times with a fire-polished silanized Pasteur pipette in Hibernate-E without Calcium containing 2% B-27, 0.01% DNaseI, 1 mg/ml BSA, and 1 mg/ml Ovomucoid Inhibitor. Undispersed pieces were allowed to settle by gravity for 1 min and the supernatant is centrifuged for 3 min at 228 g. The hippocampal pellet was resuspended in Hibernate-E containing 2% B-27, 0.01% DNaseI, 1 mg/ml BSA, 1 mg/ml Ovomucoid Inhibitor and diluted with Hibernate-E containing 2% B-27. After the second centrifugation step (5 min at 228 g), the pellet was resuspended in nutrition medium with glutamate (Neurobasal, 2% B-27, 0.5 mM glutamine, 25 μM glutamate, 1% Penicillin-Streptomycin).

### Preparation of Primary Cultures of Mouse Cerebral Cortical Astrocytes.

Primary cultures of cerebral cortical astrocytes were prepared from Swiss albino newborn mice (1-2 days old) as described [Pellerin L, Magistretti P J. Glutamate uptake into astrocytes stimulates aerobic glycolysis: a mechanism coupling neuronal activity to glucose utilization. Proc Natl Acad Sci USA. 1994 Oct. 25; 91(22):10625-9]. This procedure yields cultures that are >95% immunoreactive for glial fibrillary acidic protein.

### Cell Treatments.

Neuronal cultures were treated by direct application of compounds into the culture medium using 50-100× stock solutions glutamate or NADH. Compound A was added min prior to glutamate or NADH treatments.

### Cell Viability Assays

The MTT assay was conducted according to manufacturer's instructions (Invitrogen/Molecular Probes, Eugene, Oreg.) and was measured using a plate reader at an absorbance wavelength of 570 nm. Cell survival rate was expressed either as the absorbance values or as optical density (OD), with values calculated as % of controls.

### Statistical Analysis.

All results are presented as the mean f SEM and significance was accepted at P≤0.05 for all statistical tests. Data were analysed for statistical significance by unpaired Student t test or by one-way ANOVA. Statistically significant one-way ANOVAs were followed by a post hoc Dunnett's multiple comparison test when all groups were compared with the control group, or by a Bonferroni's multiple comparison test when comparing all pairs of groups (Prism 5.0; GraphPad).

It will be understood that the present invention has been described above by way of example only. The examples are not intended to limit the scope of the invention. Various modifications and embodiments can be made without departing from the scope and spirit of the invention, which is defined by the following claims only.

### Example 1

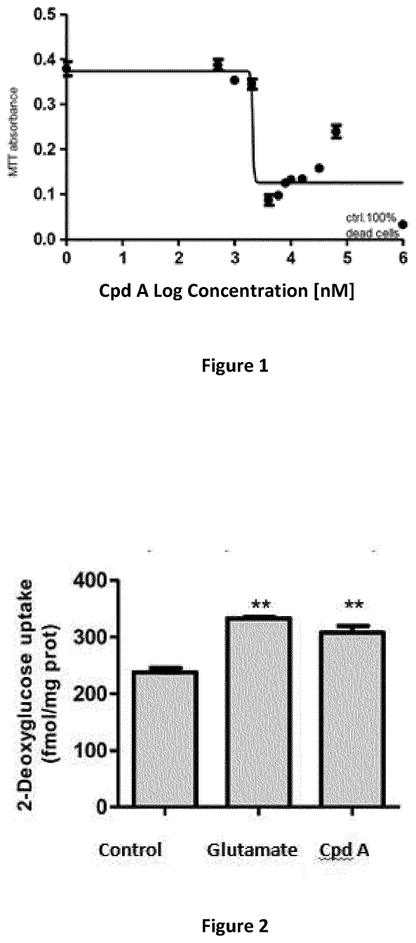

Primary neuronal culture viability in the presence of Compound A/SND118 Primary neuron culture was prepared as described and treated with Compound A/SND118 at various concentrations for 24 hours. Cellular viability was measured using MTT assay as described. Results indicated that under these conditions Compound A is not toxic up to concentrations of 10 μM (FIG. 1).

### Example 2

### Effect on Astrocytes Cultures/Activation of Astrocyte Function

Astrocytes, thought to be the predominant type of glial cell in the brain, are involved in a wide range of CNS functions, including control of blood flow, glucose metabolism, glutamate clearance, ionic homeostasis (particularly K⁺), synaptic development, and neuronal plasticity. It is well established that glucose is an obligatory fuel, critically important for many brain functions, including ATP production, oxidative stress management, and synthesis of neurotransmitters, neuromodulators, and structural components of the cell. Neuronal ATP production with astrocyte-derived L-lactate was proposed as a model of activity-dependent energy metabolism called astrocyte-neuron L-lactate shuttle (ANLS) [Pellerin L, Magistretti P J. Glutamate uptake into astrocytes stimulates aerobic glycolysis: a mechanism coupling neuronal activity to glucose utilization. Proc Natl Acad Sci USA. 1994 Oct. 25; 91(22):10625-9].

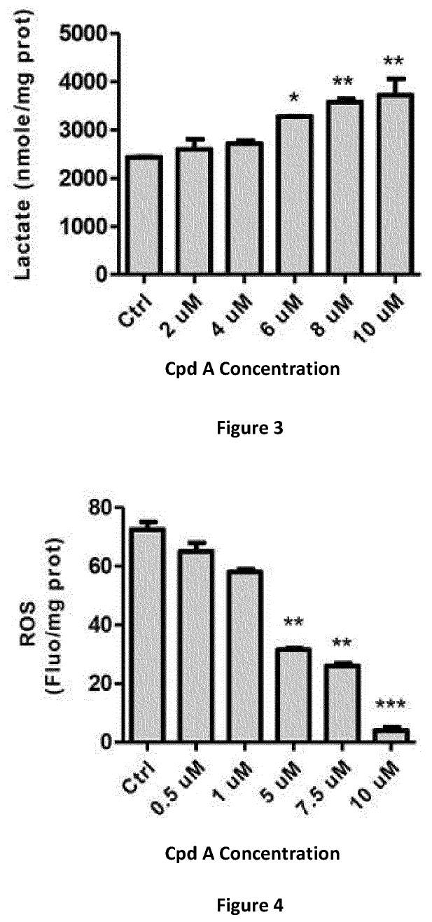

Astrocytes are emerging as having significant roles in several homeostatic processes in the brain [Zuchero J B, Barres B A. Glia in mammalian development and disease. Development 2015 142: 3805-3809]. To evaluate the potential effects of Compound A (10 μM) on astrocyte metabolism, the uptake of glucose and lactate release were measured 30 minutes after application of compounds to the culture (FIGS. 2 and 3).

Glucose utilization by cells was measured using radioactive 2-deoxyglucose, a well-established marker of glucose utilization not metabolized within cells. For the astrocytes cultures, glutamate at a concentration of 200 μM is known to increase glucose entry by about 20 to 30% and was used as a positive control.

### Experimental Methods:

In order to quantify glucose utilization by cells in the presence of Compound A/SND118, radioactive 2-deoxyglucose, a well-established marker of glucose utilization not metabolized within cells, was used. The radioactivity count is thus proportional to the transport and phosphorylation of glucose that enters into the cells. For the astrocyte cultures, glutamate is known to increase glucose entry by about 20 to 30% and was therefore used as a positive control.

Glucose uptake was measured as previously described [Allaman I. et al, (2004) Glucocorticoids modulate neurotransmitter-induced glycogen metabolism in cultured cortical astrocytes. J. Neurochem. 88, 900-908]. 2-[1,2-3H]Deoxy-D-glucose ([3H]-2-DG) (specific activity, 30-60 Ci/mmol) was obtained from ANAWA. The effect of glutamate on astrocytic glucose uptake was measured in parallel in other Petri dishes by adding glutamate 200 μM in the medium containing [3H]2-deoxyglucose for 20 min of incubation. Other Petri dishes were used to measure the portion of glucose uptake that is not linked to glucose transporter by addition of the glucose transporter inhibitor cytochalasin B (Sigma-Aldrich) 25 μM during 20 min of incubation. The fraction of glucose transported is calculated by subtracting the fraction of glucose uptake that is not inhibited by the cytochalasin B. Glucose uptake was normalized to the protein content.

### Lactate Release Assay

Lactate release into the medium was measured enzymatically by a modification of the enzymatic spectrophotometric method of Rosenberg and Rush [Rosenberg J C, Rush B F. An enzymatic-spectrophotometric determination of pyruvic and lactic acid in blood. Methodologic aspects. Clin Chem. 1966; 12(5):299-307.]. Incubations were carried out exactly as described for [3H]2DG uptake experiments except for the fact that no tracer and no phenol red (which otherwise interferes with the spectrophotometric determination of lactate) were present in the incubation medium. The reaction was terminated by collecting the supernatant on ice, while cells were treated as described above for protein determination.

### ROS Formation

ROS formation has been determined as described [Yang J et al, Lactate promotes plasticity gene expression by potentiating NMDA signalling in neurons. Proc Natl Acad Sci USA. 2014. 111(33):12228-33] using a H2DCF-DA kit (ThermoFisher) as recommended by the manufacturer. Briefly, astrocytes cultures were washed twice with HBSS and incubated for 60 min in 50 μM in HBSS at 37° C. and 5% CO₂ in the presence of the dye. After two washing steps with prewarmed HBSS the cells were treated with increasing concentrations of 100 μL Compound A at 37° C. and 5% CO₂. Fluorescence intensity was measured after 2 h from the same plate using a fluorescence microplate reader (Safire 2; Tecan) at an excitation wavelength of 485 nm and an emission wavelength of 528 nm.

### Measurements of Cellular ATP/ADP Ratio

ATP content was measured enzymatically as previously described [Lambert H P et al, Control of Mitochondrial pH by Uncoupling Protein 4 in Astrocytes Promotes Neuronal Survival, 2014 The Journal of Biological Chemistry 289, 31014-31028] using a luciferase assay, the CellTiter-Glo Luminescent cell viability assay (Promega). Astrocytes grown on multiplate of 48 wells were rinsed and incubated 1 h at 37° C. in an atmosphere containing 5% CO₂ and 95% air in DMEM (D5030; Sigma-Aldrich) containing 44 mm NaHCO₃ and 2 mm glucose. At the end of the incubation, medium was removed, and 200 μl of Tricine buffer solution (40 mm Tricine, 3 mm EDTA, 85 mm NaCl, 3.6 mm KCl, 100 mm NaF, and 0.1% saponin (84510; Sigma-Aldrich), pH 7.4) was put in each well. Cells were lysed by saponin effect and by pipetting. Each sample was divided for ATP measure and for ATP+ADP measure. 90-μl aliquots were distributed in a black-walled 96-well type microplates (PerkinElmer Life Sciences). For the ATP+ADP measure, 10 μl of converting solution (100 mm Tricine, 100 mm MgSO₄, 25 mm KCl, 1 mm phosphoenolpyruvate, and 100 units/ml pyruvate kinase), pH 7.75, was added in each well, whereas the same solution without phosphoenolpyruvate and pyruvate kinase was added to the samples for ATP measure. An incubation of 5 min at room temperature was performed before adding 10 μl of MgCl₂ solution (4 mm Tricine and 100 mmMgCl₂). Finally, 100 μl of CellTiter-Glo reagent (G7571; Promega) was added, and luminescence was immediately detected with a luminometer (Safire 2; Tecan). Luminescence was measured in a kinetic way determined by 20 readings at intervals of 1 min. Luminescence read at the plateau were taken to calculate the ATP/ADP ratio.

### NAD/NADH Assay.

Cycling assays for nicotinamide adenine dinucleotides was performed as described [Yang J, et al, Lactate promotes plasticity gene expression by potentiating NMDA signaling in neurons. Proc Natl Acad Sci USA. 2014. 111(33):12228-33]. Briefly, cells were rinsed two times with ice-cold PBS, harvested in 400 μL ice-cold carbonate-bicarbonate buffer (100 mM Na₂CO₃ and 20 mM NaHCO₃ containing 10 mM nicotinamide to inhibit NADase), and frozen at −80° C. Cell membranes were lysed by heat shock in a 37° C. water bath and immediately chilled on ice. Extracts were centrifuged at 12,000×g for 30 min at 4° C. and half of the supernatant was heated at 60° C. for 30 min to denature NAD. Twenty-five microliters of the heated extract (containing NADH only), 100 μL of the unheated extract (containing NAD and NADH), and 50 μL of standards of known NADH (Roche) concentrations (ranging from 0.0625 to 1 μM) dissolved in carbonate bicarbonate buffer were loaded onto a 96-well microplate along with blanks (carbonate-bicarbonate buffer). Volumes were adjusted to 100 μL with carbonate-bicarbonate buffer and 150 μL of a reaction buffer was added into each well. Reaction buffer contained 133 mM bicine, 5.33 mM EDTA, 0.56 mM methylthiazolyldiphenyl-tetrazolium bromide, 2.11 mM phenazine ethosulfate, 0.67 M ethanol, and 40 U/mL alcohol dehydrogenase (Sigma-Aldrich). The absorbance was followed spectrophotometrically at 560 nm every 15 s over a 5-min period (Safire 2; Tecan). Blank values were subtracted from all samples and NAD amounts were calculated by subtracting NADH values from total NAD+NADH values.

### Results:

As presented in FIGS. 2-4, SND118 increased the uptake of deoxyglucose in the same range as glutamate control, increased the release of L-lactate and led to a decrease in ROS accumulation.

FIG. 2; glucose uptake: control=vehicle; Glutamate=glutamate (200 PM); Cpd A=SND118 (10 μM).

FIG. 3: Lactate release in the presence of various concentrations of Cpd A/SND118.

FIG. 4: ROS accumulation in the presence of various concentrations of Cpd A/SND118.

ROS can influence multiple aspects of neural differentiation and function, including the survival and the plasticity of neurons, the proliferation of neural precursors, as well as their differentiation into specific neuronal cell types. In the mammalian central nervous system, reactive oxygen species (ROS) generation is counterbalanced by antioxidant defenses. When large amounts of ROS accumulate, antioxidant mechanisms become overwhelmed and oxidative cellular stress may occur [Samina S. Oxidative Stress and the Central Nervous System. J Pharmacol Exp Ther 360:201-205, January 2017]. Therefore, ROS are typically characterized as toxic molecules, oxidizing membrane lipids, changing the conformation of proteins, damaging nucleic acids, and causing deficits in synaptic plasticity. High ROS concentrations are associated with a decline in cognitive functions, as observed in some neurodegenerative disorders and age-dependent decay of neuroplasticity.

To assess the effect of Compound A on ROS accumulation in primary neuron cultures, the cells were treated with various concentration of Compound A for 2 hours and ROS was measured as described (FIG. 4).

The decreased ROS formation due to the potential anti-oxidant effect of Compound A.

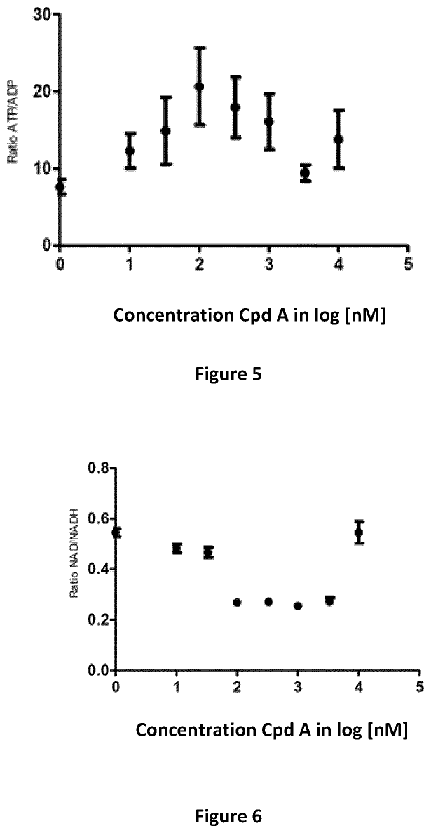

Changes in the ratio of ATP to ADP content is a key indicator of cells' bioenergetic status, with rising ATP/ADP ratios indicating increased energy reserves, and declining ATP/ADP ratios indicating lower energy supplies (or increased ATP use). As shown in FIG. 5, ATP/ADP ratios trended higher at most doses of Compound A analyzed.

An increased production of ATP In the presence of 100 nM of Compound A following treatment of the neuron culture for 30 min.

The fact that Compound A/SND118 promotes glycolysis was confirmed by the observation of an increased production of ATP and NADH after 30 min in the presence of the compound, as presented in FIGS. 5 and 6.

FIG. 5: SND118; ATP/ADP ratio; x axis concentration of SND118 in log [nM].

FIG. 6: SND118; NAD/NADH ratio; x axis concentration of SND118 in log [nM].

### Example 3

### Induction of Immediate-Early Genes Linked to Neuronal Plasticity

In the brain, neuronal gene expression is dynamically changed in response to neuronal activity. In particular, the expression of immediate-early genes (IEGs) such as egr-1, c-Fos, and Arc is rapidly and selectively upregulated in subsets of neurons in specific brain regions associated with learning and memory formation [Minotohara Keiichiro, Role of Immediate-Early Genes in Synaptic Plasticity and Neuronal Ensembles Underlying the Memory Trace. Front Mol. Neurosci. 2015; 8: 78]. IEG expression has therefore been widely used as a molecular marker for neuronal populations that undergo plastic changes underlying formation of long-term memory.

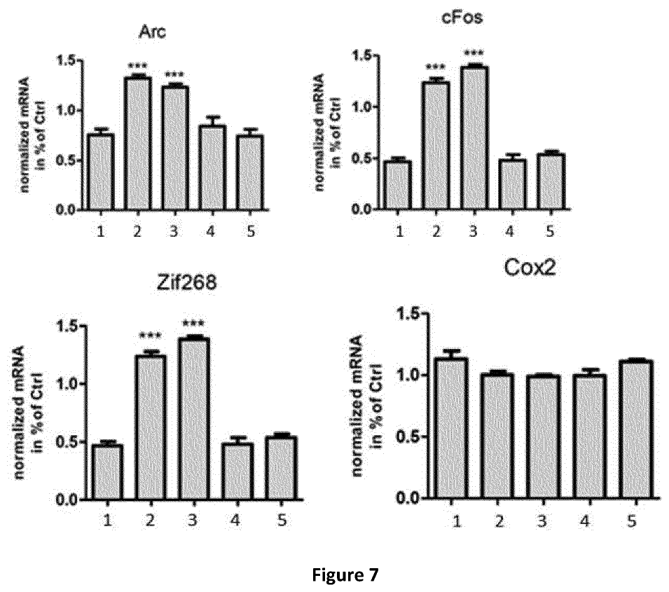

The effect of Compound A or derivative thereof on the mRNA expression of genes related to plasticity (Arc, cFos, and Zif268) was determined as described. Cox (cytochrome oxidase) was used to evaluate if the derivative changes expression of mitochondrial genes. As shown in FIG. 7, plasticity gene expression is increased in the presence of the Compound A/SND118 while Cox is unaffected.

### Experimental Method

### Quantitative PCR.

Determination of gene expression was performed as previously described [Yang J, et al, Lactate promotes plasticity gene expression by potentiating NMDA signalling in neurons. Proc Natl Acad Sci USA. 2014. 111(33):12228-33]. Total RNA was isolated from cultured cells using Nucleospin RNA II kit (Macherey-Nagel) according to the manufacturer's instructions. The first strand of cDNA was synthesized from 100 ng of total RNA (60 min at 37° C. followed by 5 min at 95° C.) using a high-capacity RNA to cDNA reverse transcription system (Applied Biosystems). One-twentieth of the resulting cDNA was amplified by quantitative PCR (qPCR) with an ABI Prism 7900 system (Applied Biosystems). The PCR mix was composed of 6 ng of cDNA, 300 nM of forward and reverse primers in 10 μL of ix SYBR-Green PCR MasterMix (Applied Biosystems). Primer sequences were designed using Primer Express 3.0 software (Applied Biosystems) and oligonucleotides were synthesized by Microsynth.

### Results

The effect of SND118 derivative on the mRNA expression of genes related to plasticity (Arc, cFos, and Zif268) was determined as described. The Cox (cytochrome oxidase) was used to evaluate if the derivative changes expression of mitochondrial genes. As shown in FIG. 7, plasticity gene expression is increased in the presence of the Compound A/SND118 while Cox is unaffected.

FIG. 7: Plasticity gene expression 1—control; 2—SND118 10 μM 1 h treatment; 3—SND118 10 μM 2 h treatment; 4—SND118 1 μM 1 h treatment; 5—SND118 1 μM 2 h treatment.

In vitro exposure of primary brain cell cultures to Compound A led to an increase in glucose uptake and an increase in lactate release suggesting an effect on the Astrocyte-to-Neuron Lactate Shuttle (ANLS) [Pellerin L, Magistretti P J. Glutamate uptake into astrocytes stimulates aerobic glycolysis: a mechanism coupling neuronal activity to glucose utilization. Proc Natl Acad Sci USA. 1994 Oct. 25; 91(22):10625-9] which postulates that in times of increased neuronal activity, and thus energy demand, astrocytes take up blood glucose via their particularly well-positioned end feet on capillaries and convert this glucose to lactate. The induction of the IEG Arc, C-Fos and Zif 268 suggest a function of Compound A in synaptic plasticity, and thus in memory and learning processes.

### Example 4—Activation of TrkB Receptor

Experimental Method:

On the day of preparation (DIV1) cortical neurons were seeded on poly-D-lysine pre-coated 6-well plates at a density of 1.25*10\hat{ }6 cells per well and cultured at 37° C.; 95% humidity and 5% CO₂ until DIV8 with a half medium exchange on DIV4-6. On DIV10 cells are treated with test item TI (SND118) and control Rh (7,8DHF) at different concentrations for 15 min. The experiment was carried out with n=6 technical replicates per condition, vehicle treated cells served as control. Cells were lysed in 150 μL cold RIPA buffer [50 mM Tris pH 7.4, 1% Nonidet P40, 0.25% Na-deoxy-cholate, 150 mM NaCl, 1 mM EDTA supplemented with freshly added 1 μM NaF, 0.2 mM Na-ortho-vanadate, 80 μM Glycerophosphate, protease (Calbiochem) and phosphatase (Sigma) inhibitor cocktail.

Phosphorylation of the TrkB receptor was detected using the following rabbit anti-Tyr specific antibodies: anti-TrkB Y515, Y706/707 and Y816 and compared with total TrkB detected with anti-TrkB antibody (Abcam). Antibody dilutions and protein amounts were optimized for signal specificity.

Automated separation and immunostaining of total and phospho TrkB was carried out using a capillary-based immunoassay, WES™ (Proteinsimple®). Samples were applied to a 25 capillary cartridge with a 2 to 440 kDa matrix, at an optimized total protein concentration. Sample loading, separation, immunoprobing, washing, detection and quantitative data analysis were performed automatically by WES™ Western system (Compass software V 4.0.0). The areas under the curve were used for the analysis and the phosphorylated versus vehicle or total TrkB signal ratio was calculated and used for statistics.

### Results:

SND118 at a concentration of 1 and 2 μM induced statistically significant phosphorylation of the TrkB receptor at sites Tyr 515 and Try 816, while 7,8DHF induced a lower phosphorylation, which did not reach statistical significance. While 2 μM conc of SND118 increased slightly the phosphorylation at Tyr residue 706/707, neither the derivative nor the 7,8DHF reached statistical significance versus the vehicle. However, when the signal of phospho Tyr706/707 was normalized to the TrkB, SND118 led to a significant increase. The results are presented in Table 1.

P-value results from One way ANOVA followed by Dunnett's multiple comparisons test. ns=not significant, p-values between 0.05 and 0.3 are given as numbers. *p<0.05; ** p<0.01; *** p<0.001.

TABLE 1
Analysis of TrkB phosphorylation

| | SND118 | SND118 | 7,8 DHF | 7,8DHF |
|---|---|---|---|---|
| | 2 μM | 1 μM | 2 μM | 1 μM |
| Tyr 515 | **↑ | *↑ | ns | 0.22 |
| Tyr 515/TrkB | **↑ | **↑ | 0.09 | ns |
| Tyr 706/707 | 0.09 | ns | ns | Ns |
| Tyr 706/707/TrkB | **↑ | 0.14 | 0.29 | *↑ |
| Tyr 816 | *↑ | ns | ns | 0.12 |
| Tyr 816/TrkB | ***↑ | ns | ns | 0.09 |

### Example 5—Neuron Protection from Glutamate Injury

Increasing evidences suggest that glutamate and mitochondria are two prominent players in the oxidative stress (OS) process that underlie AD and PD. Glutamate is an important neurotransmitter in neurons and glial cells and is strongly dependent on calcium homeostasis and on mitochondrial function. Excitotoxicity, the process by which overactivation of excitatory neurotransmitter receptors leads to neuronal cell death supports a key role for massive Ca²⁺ influx through the NMDA receptor (NMDAR) channel as a trigger of glutamate neurotoxicity [Schinder A F et al, Mitochondrial Dysfunction Is a Primary Event in Glutamate Neurotoxicity. J Neurosci. 1996 Oct. 1; 16(19): 6125-6133]. Given that excessive Ca²⁺ accumulation in mitochondria uncouples electron transfer from ATP synthesis mitochondria is considered a link between elevation of [Ca²⁺] and glutamate neurotoxicity.

### Experimental Method:

Cortices were harvested from E19 rat embryos and dissociated enzymatically and mechanically. Dissociated cells were plated in poly-D-lysine coated imaging plates (384 wells), in 70 μL of neuronal growth medium (Neurocult Neuronal Basal medium+SM1 neuronal supplements+L-glutamine+HEPES). Cells were incubated at 37° C., 5% CO₂ and half of the medium was changed twice per week.

For Calcium measurement, the cells were cultured for 10 to 14 days in vitro, the growth medium was discarded and replaced by 25 μL of a calcium probe in a saline solution (containing 1.5 mM Calcium) for 30 min at 37° C./5% CO₂. 5 μL of calcium probe in a saline solution with or without the test substances at 6× concentrated was added in the wells for the pre-treatment step. The wells were further incubated 30 min at 37° C./5% CO₂

For calcium measurement, at the end of the pre-treatment, glutamate was prepared at 6× concentrated (6 μL on top of the 30 μL). The final vehicle concentration in all conditions was adjusted. Basal calcium levels was measured for 1 minute before automated addition of the compounds or controls while recording. Intracellular calcium signals was further recorded for 5 to 10 minutes at a sampling rate of around 1 point per second. Each experimental condition was tested in quadruplicate wells.

For mitochondrial membrane potential measurement (MMP) neurons were cultured 10 to 14 days in vitro, the growth medium was discarded and wells were washed with 50 μL of extracellular saline. Saline was removed and replaced by 25 μL of staining solution (rhodamine 123 in a saline solution containing 1.5 mM Calcium, pyruvate and verapamil). The cells were incubated 30 min at 37° C./5% CO₂. 5 μL of a staining solution with or without the test substances at 6× concentrated was added in the wells. Cells were incubated another 30 min at 37° C./5% CO₂.

For MMP measurement, at the end of the pretreatment step, the wells were washed with 50 μL of saline solution+verapamil+pyruvate. Then, 30 μL of saline+verapamil was added.

Glutamate was prepared 6× concentrated (6 μL on top of the 30 μL) in saline or saline+test compounds 1× concentrated depending on the conditions. The final vehicle concentration in all conditions was adjusted. Basal MMP levels were measured for 1 minute before automated addition of the compounds or controls while recording. MMP signals were further recorded for 30 minutes to 1 hour. Each experimental condition was tested in quadruplicate wells.

### Results:

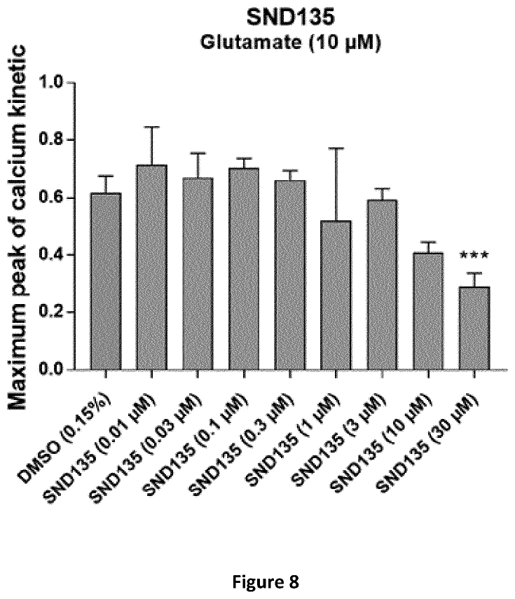

The results are presented in FIGS. 8 to 13.

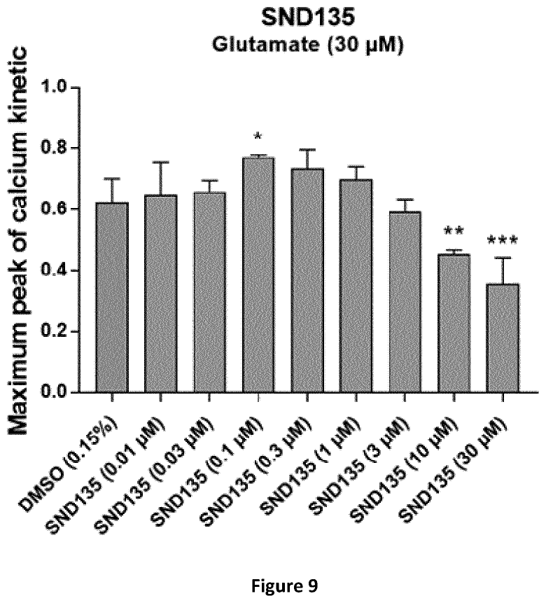

FIG. 8 relates to a glutamate concentration of 10 μM and shows the maximum peak of calcium kinetic for SND135 at a range of concentrations. FIG. 9 relates to a glutamate concentration of 30 μM and shows the maximum peak of calcium kinetic for SND135 at a range of concentrations. SND135 at concentrations of 10 and 30 μM decreases calcium release induced by the effects of glutamate at 10 and 30 μM. Statistical analysis by one way Anova followed by Dunett's test (against vehicle control) (*=p<0.05; ** p<0.01; *** p<0.0010

Rhodamine 123 is a cationic fluorescent dye that is used to specifically label respiring mitochondria. The dye distributes according to the negative membrane potential across the mitochondrial inner membrane. Loss of potential will result in loss of the dye and, therefore, the fluorescence intensity.

Our studies have shown that SND derivative restores mitochondria potential increased by the addition of glutamate.

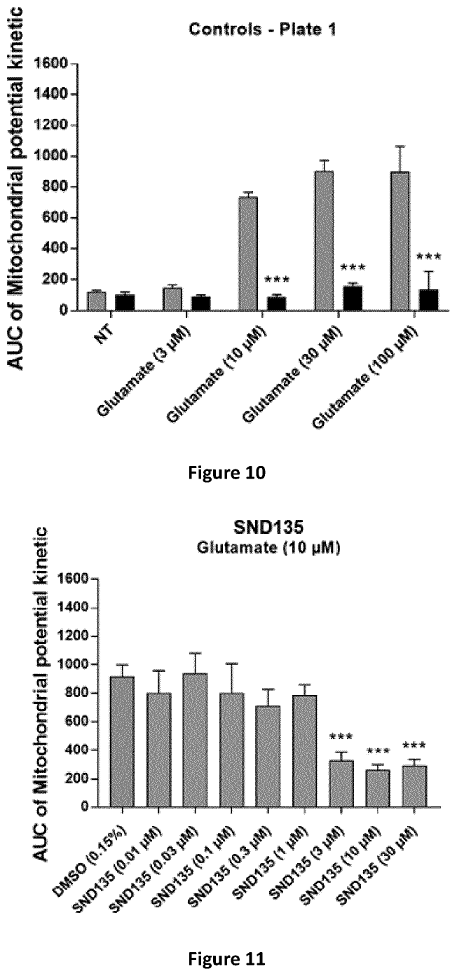

FIG. 10 shows that glutamate increases mitochondria potential, which is restored by the control compound [(+)-5-methyl-10,11-dihydroxy-5H-dibenzo(a,d)cyclohepten-5,10-imine] also known as dizocilpine hydrogen maleate (MK801).

The left-hand bar of each pair is vehicle control DMSO 0.15%; the right-hand bar of each pair is MK801 10 μM.

FIG. 11 shows the effect of SND135 on the mitochondria potential at a concentration of glutamate of 10 μM in comparison to vehicle.

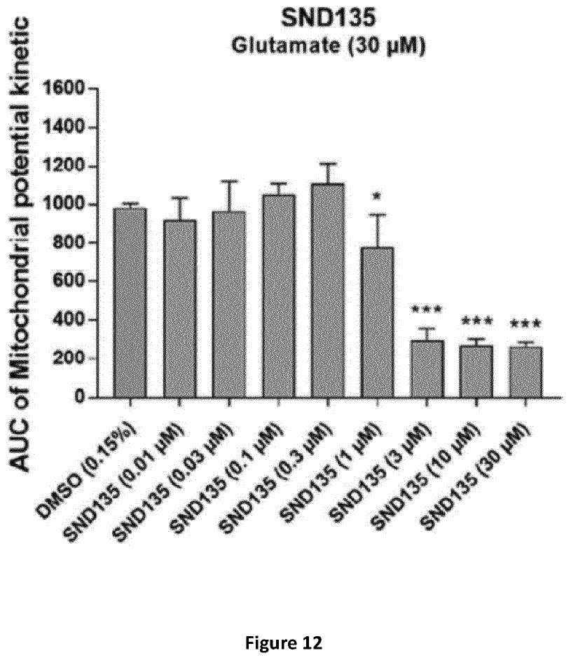

FIG. 12 shows the effect of SND135 on the mitochondria potential at a concentration of glutamate of 30 μM in comparison to vehicle.

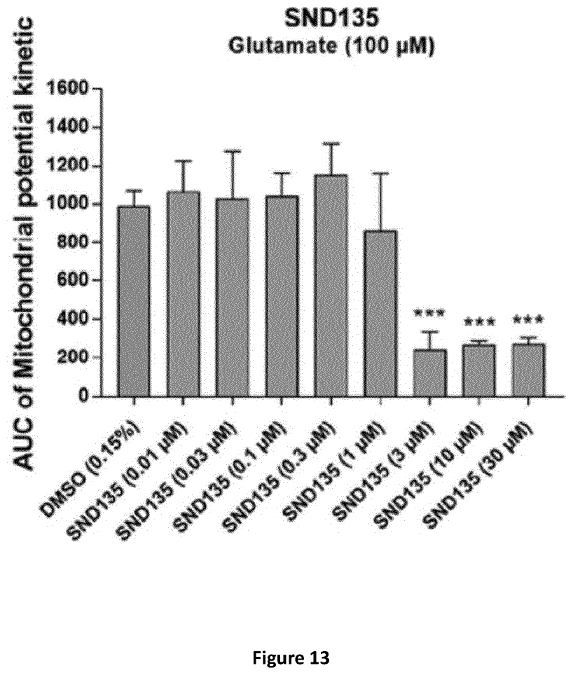

FIG. 13 shows the effect of SND135 on the mitochondria potential at a concentration of glutamate of 100 μM in comparison to vehicle.

SND135 at concentrations between 3 and 30 μM decreases mitochondria potential induced by the effects of glutamate at concentrations between 10 and 100 μM. Statistical analysis by one way Anova followed by Dunett's test (against vehicle control) (*=p<0.05; ** p<0.01; *** p<0.0010).

SND135 over the dose-range 3-30 μM protected against glutamate concentrations of 10, 30 and 100 μM by decreasing mitochondrial staining. Calcium release was also decreased by SND135 at 30 μM against glutamate at 30 μM. These effects suggest SND135 presents neuroprotective activity against excitotoxicity.

### Example 6—Protection of Organotypic Brain Slices from Iodoacetic Acid Injury

Similar to ischemia in vivo, iodoacetic (IAA) treatment of brain cells causes excessive ROS generation which may lead to mitochondrial membrane depolarization to induce the apoptosis cascade, which results in functional and structural damage to neuronal cells. Therefore, neuroprotective agents that scavenge free radicals and maintain mitochondrial function are considered a potential therapeutic strategy for treating ROS-related disorders, especially ischemic stroke.

### Experimental Method:

Preparation of the organotypic brain slices were performed by decapitation of the P9/P10 mouse pups, removing skin and skull and immersing brains in slicing medium (Opti-MEM 1, 20 μM glucose). Brains were hemisected and hippocampi were isolated. Hippocampi were placed on the cutting disc of a McIlwain Tissue Chopper. 300 μM thick hippocampal slices were chopped transversely. A number of 4 slices per hippocampus were placed on porous (0.4 μM) transparent membrane inserts (Millipore) and incubated for 1 h on ice in HBSS containing 10 mM glucose. Afterwards inserts were transferred to fresh 24-well plates containing 250-300 μl culture medium (50% MEM/EBSS, 25% horse serum, 25% CMF-HBSS, 25 mM glucose, 0.5% pen/strep). Slices were maintained at 37° C. and 5% CO₂.

On DV15, brain slices were pretreated with SND derivatives test compounds and the reference 7,8DHF at various concentrations (between 1 and 30 PM) for 30 min, followed by the addition of iodoacetic acid (IAA) at 250 μM for 110 min, when cell survival, MMP and toxicity were measured.

Cell survival was measured by the MTT assay. For the determination of mitochondrial membrane potential, treated organotypic brain slices were loaded with the mitochondrial fluorescent dye, tetramethylrhodamine methyl ester (TMRM) at a final concentration of 100 nM in PBS and incubated for 45 min at 37° C. The TMRM containing solution was aspirated and slices were transferred into new well plates containing the appropriate amount of PBS. Fluorescence was measured with a plate-reader (Cytation 5) using wavelengths of excitation and emission of 548 and 574 nm, respectively (area scan). Values were calculated as percent of control values (vehicle control). Toxicity of the treatment was measured by LDH. No toxicity was observed.

Results:

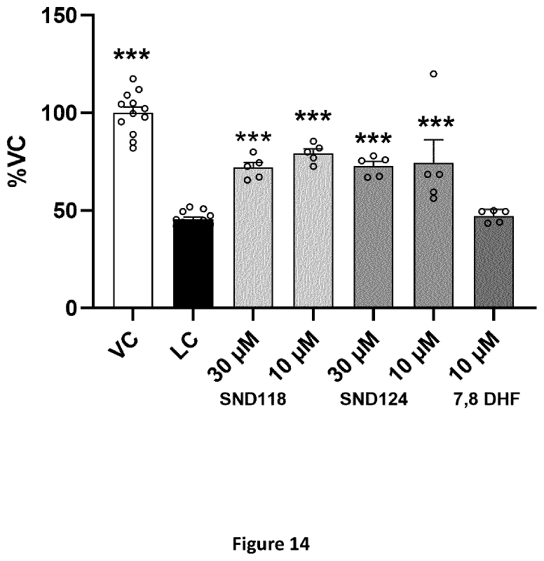

FIG. 14 shows that SND118 and SND 124 restore MMP decreased by IAA lesion. VC=vehicle control; LC=lesion control.

SND118 at concentrations of 10 and 30 μM and SND124 at 30 μM statistically increase cell survival and rescued mitochondria function from the injury induced by IAA. 7,8 DHF at the concentration of 10 μm has no effect on protecting brain slices from IAA lesion.

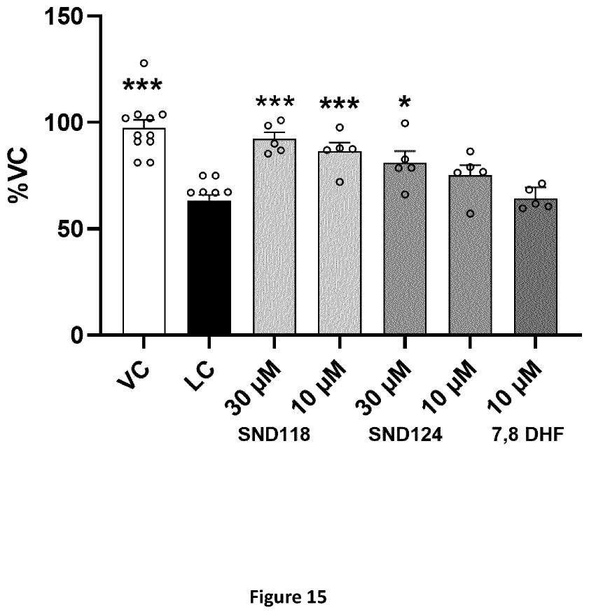

FIG. 15 shows that SND118 and SND124 increase cell survival upon IAA lesion.

### Example 7—Protection of Neurons from MPP+Injury

1-methyl-4-phenylpyridinium (MPP⁺) is widely used in vitro to simulate the damage of DAnergic neurons seen in PD. MPP⁺ induces oxidative stress through interfering with oxidative phosphorylation in mitochondria, leading to the damage and death of dopaminergic neurons.

### Experimental Method:

Mouse cortical neurons were prepared as described. At DIV8 cells were treated with the test and control MK801 compounds for 1 h, before the addition of MPP+(Sigma, D048) at a final concentration of 100-200 μM. The experiment was carried out with n=6 technical replicates per condition, vehicle and MPP+ alone controls were included. After 8 h determination of ROS was conducted and after 24 h of MPP+ lesion on DIV9 cells were subject to the apoptosis measurement. Apoptosis was determined using YO-PRO™-1 Iodide (Invitrogen; Y3603). Part of supernatant of the cultivated cells was removed and 10 μL of a 50 μM YO-PRO 1 solution in PBS is added to the remaining 90 μL to result in a final concentration of 5 μM YO-PRO 1 in well. Incubation for 15 min in the incubator at 37° C. was performed (light protected) followed by discarding the supernatant. 140 μL PBS was added to each well and the fluorescence was measured using a plate-reader (Cytation 5, BioTek) at excitation wavelength 485 nm to Emission wavelength 535 nm.

ROS generation was measured with Abcam's DCFDA-Cellular Reactive Oxygen Species Detection Assay (ab 113851). The kit uses the cell permeant reagent 2′,7′-dichlorofluorescin diacetate (DCFDA), a fluorogenic dye, which after its diffusion into the cell is deacetylated by cellular esterases to a non-fluorescent compound, which is later oxidized by ROS into 2′,7′-dichlorofluorescein (DCF). DCF is a highly fluorescent compound. The assay was performed as described in the manufacturer's protocol. Briefly, after washing cells once in ix buffer, they were incubated with 25 μM DCFDA for 45 minutes at 37° C. Cells were then incubated with the test items and lesion for 8 h and thereafter the fluorescent signal was measured at 485 nm/535 nm (Cytation 5, BioTek).

### Results:

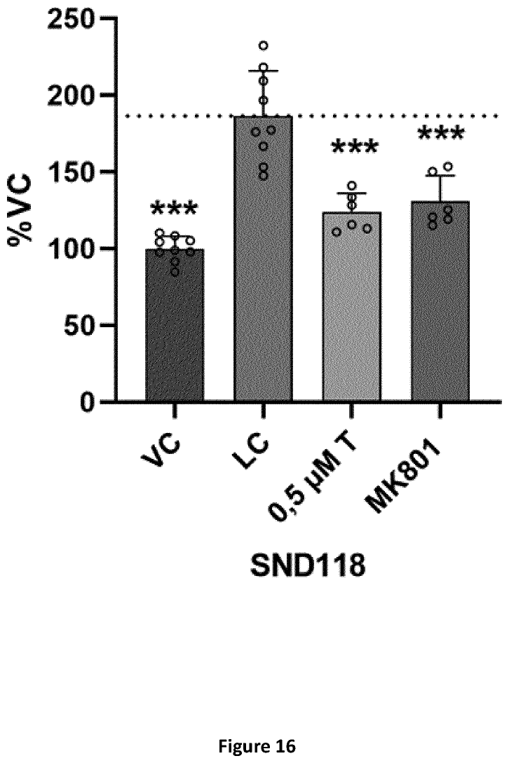

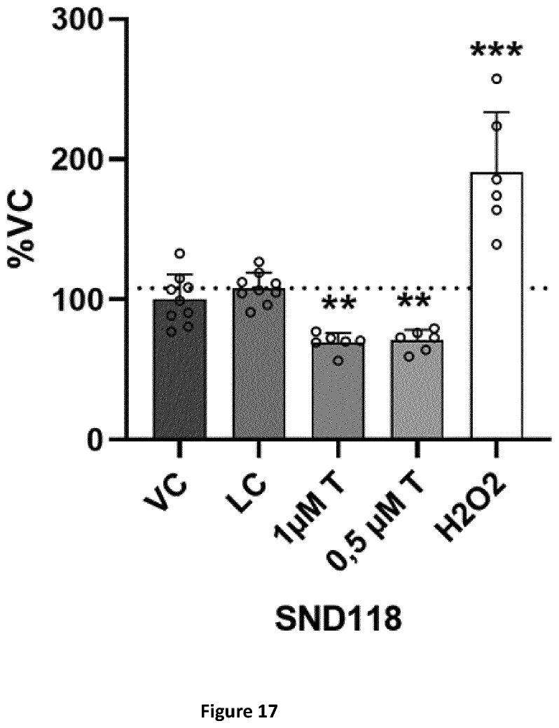

SND118 at concentrations of 0.5 and 1 μM significantly inhibited apoptosis induced by MPP+ and decreased the level of ROS in the cortical neurons. Measurement of MPP+ induced apoptosis (FIG. 16) and ROS (FIG. 17) in primary neurons, treated with test and reference item in combination with MPP+ lesion on DIV8 for 24 h for apoptosis and 8 h for ROS determination. Cells were then assayed using YOPRO and DCFDA reagent, respectively, according to the manufacturer's instructions. Data are displayed as % of the vehicle control (% VC) as bar graphs (mean+SD) and data points are shown as dots. One way ANOVA followed by Dunnett's multiple comparisons test compared to the lesion control (LC). ** p<0.01; *** p<0.001

### Example 10—Protection of Microglia Cells from Inflammation

Under pathological conditions, activated microglia release pro-inflammatory mediators, including nitric oxide (NO), prostaglandin E₂ (PGE₂), reactive oxygen species (ROS) and pro-inflammatory cytokines [Loane, D. J., Byrnes, K. R. Role of microglia in neurotrauma. Neurotherapeutics 7, 366-377 (2010).]. The overproduction of these inflammatory mediators and cytokines causes severe forms of various neurodegenerative diseases, such as Alzheimer's disease (AD), cerebral ischemia, multiple sclerosis and trauma.

To test if SND derivatives will protect microglia cell line BV2 from LPS-induced inflammatory markers, the compounds were assayed in an in vitro well-established assay.

### Experimental Method:

The murine microglial cell line BV-2 was grown in DMEM medium supplemented with 10% FCS, 1% penicillin/streptomycin and 2 mM L-glutamine (culture medium). For LPS stimulation assay, 10000 BV-2 cells per well (uncoated 96 well plates) were plated and after 24 hours, medium was changed to serum-free treatment medium (DMEM, 2 mM L-glutamine) and cells were maintained in treatment medium for the remaining culture period. 1 h after changing cells to treatment medium, the test items were added 1 hour before LPS stimulation (Sigma-Aldrich; L6529; 1 mg/ml stock in ddH₂O, final concentration in well: 500 ng/ml (dilutions in medium)). Cells treated with vehicle, cells treated with LPS alone, as well as cells treated with LPS plus reference dexamethasone at 10 μM item served as controls.

Following 24 h of stimulation, cell supernatants were collected for the NO, and cytokine measurements. Levels of 2 cytokines (TNF-α, IL-6) were measured by an immunosorbent assay (U-PLEX Custom Human Cytokine, Mesoscale Discovery) according to the instructions of the manufacturer and evaluated in comparison to calibration curves provided in the kit. NO assay for the evaluation of nitrosative stress was a colorimetric assay using a diazotization reaction using Griess reagent (N-2-Aminoethyl-1-naphthylamine dihydrochloride, Sigma, Nr. G4410). 100 μl of cell culture supernatant was transferred to clear 96-well plates and 100 μl of a 40 mg/ml Griess reagent solution was added; the mixture was incubated for 15 minutes at room temperature protected from light. Absorbance was measured at 570 nm. Nitrosative stress was evaluated in the study samples in comparison to a NaNO₂ standard curve. Results were given as pg per ml. All experiments were performed in n=6 technical replicates for all groups.

### Results:

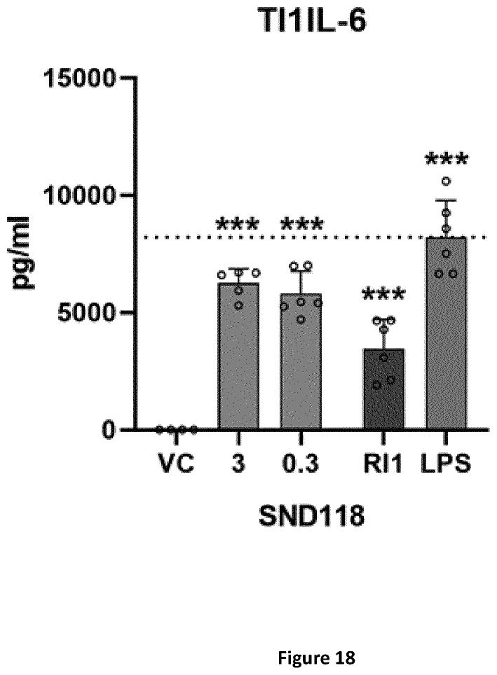

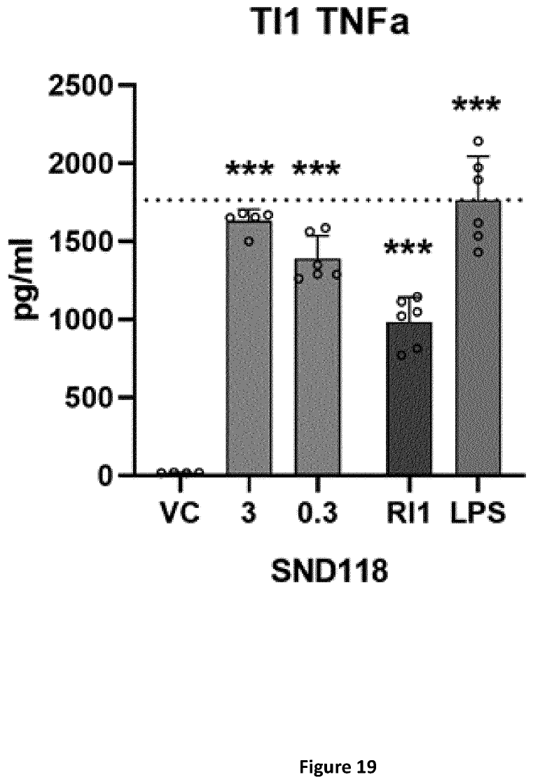

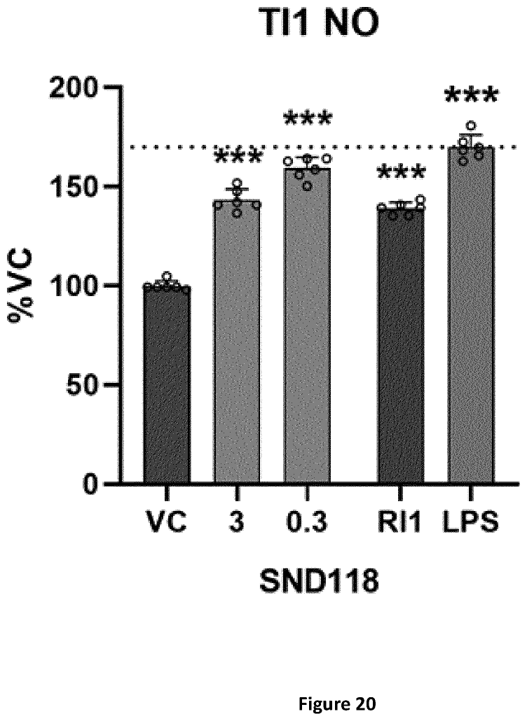

FIGS. 18-20 show measurement of inflammatory cytokines and NO in BV2 cell line in the presence of test and control treatment. VC—vehicle control; RI₁-dexamethasone at 10 μM. One way ANOVA followed by Dunnett's multiple comparisons test compared to the LPS control (LPS). * p<0.05; ** p<0.01; *** p<0.001. SND118 at concentrations between 0.3 and 3 μM significantly decreased the levels of the inflammatory cytokines IL-6 and TNF-α and NO produced by the microglia cell line in response to LPS stimulation.

### Example 11—In Vitro Inhibition of Monoamine Oxidase Type A (MOA-A) Enzyme

Recently, the involvement of type A MAO (MAO-A) in neuronal death has been shown by upregulation MAO-A expression in cellular models. MAO-A knockdown (KO) with short interfering (si)RNA protects neuronal death from apoptosis [Naoi M, Type A and B monoamine oxidase in age-related neurodegenerative disorders: their distinct roles in neuronal death and survival. Curr Top Med Chem. 2012; 12(20):2177-2188.]

### Experimental

BioVision's MAO-A inhibitor screening kit (BioVision Cat no. K796) was used to assess inhibitory effects of the test items (TIs) on MAO-A in a fluorescent assay.

The assay was carried out according to the provided protocol: TIs were diluted to 10× with MAO-A Assay Buffer before use. 10 μl of test inhibitor (S), working solution of Inhibitor Control (IC; Clorgyline, 1 μM final in the well) and MAO-A Assay Buffer (Enzyme Control; EC) were added into assigned wells. 50 μl of diluted MAO-A Enzyme Solution was added to each well and incubated for 10 min at RT. To check the possible inhibitory effect of TIs on Developer, one well with TI was prepared parallel and incubated with 50 μl of a H₂O₂ mix instead of the MAO-A Enzyme Solution.

The reaction was started by adding 40 μl of the prepared substrate mix. Measurement of the fluorescence (Ex/Em=535/587 nm) was done kinetically at 25° C. for 10-30 min. Two time points (t1 and t2) in the linear range of the plot were chosen for further calculations (2 min and 6 min). The slope for all Sample Compounds [S], Enzyme Control [EC], Vehicle Control [VC] and Background Control [BC] were calculated by dividing the net ARFU (RFU2-RFU1) values by the time Δt (t2−t1). The slope obtained for the Background Control reaction was subtracted from the [S], [EC] and [VC] values. The VC was used to calculate the relative inhibition according to the following formula: % Relative Inhibition=(Slope of [VC]−Slope of [S])/Slope of [VC]×100

### Results:

SND118 inhibits the activity of MAO-A in vitro as shown in Table 2.

TABLE 2
IC₅₀ values in vitro inhibition of MOA-A enzyme

| Compound | IC₅₀ (μM) |
|---|---|
| SND118 | 3-2 |
| SND124 | 20 |

### Example 12—In Vitro Properties of Compounds

Poor metabolic properties are a major barrier to pre-clinical and clinical development. Short-lived compounds may require excessively regular dosing to maintain a concentration in the bloodstream or the target tissue that is sufficient to elicit a therapeutic effect. In vitro metabolic screening provides a cost-effective and efficient strategy to evaluate compound metabolism during stages of discovery. To this end, the SND derivatives were tested in a panel of in vitro ADME-Tox assays to assess their properties.

### Experimental Methods:

### Cytotoxicity

Cryopreserved human hepatocytes from pooled donor lot (Bioreclamation IVT) were seeded on collagen I coated 96-well plates (Corning Biocoat) at 0.55×10⁵ cells per well in 120 μl InVitroGRO™ CP medium (BioIVT), including additives Torpedo Antibiotic mix (BioIVT). After cell attachment (4-6 hours post seeding) cell culture medium was replaced with fresh medium and incubated for 72 hours at 37° C. under 5% CO₂. Thereafter, hepatocytes were exposed to test or control compounds in 100 μl of InVitroGRO™ HI medium, including additives Torpedo Antibiotic mix at concentrations presented below. Cytotoxicity and cell viability were evaluated based on LDH release in medium and ATP content in the cells after 24 hours exposure phase.

### Metabolic Stability in Liver and Intestine Microsomes

Sample type: pooled liver or intestine microsomes;
Species: CD1-mouse (male), human (mixed gender)
Time points: 0, 10, 20, 40, 60 min cofactors, and negative control;

### Concentration: 1 μM;

Protein content: 0.5 mg/ml:
Replicates: 2 with cofactors, 1 without cofactors;
Cofactors: NADPH (1 mM)+UDPGA (1 mM)+15 μg/ml alamethicin
Buffer: 0.1 M phosphate buffer pH 7.4, 2 mM MgCl₂;
Spiking solvent: 50% DMSO ( 1/100 to incubation);
Quenching solvent: 2-fold volume of 75% ACN,
Control: midazolam disappearance rate

The study compounds were incubated with liver or intestine microsomes as specified above. The collected samples were stored at −20 C until thawed at room temperature, centrifuged and analyzed as presented below.

The samples were analysed by UPLC/HR-MS (with data dependent MS/MS mode) to monitor substrate depletion (and later optionally metabolite formation). The analytical method was optimised by using the parent compound for optimum chromatographic properties (peak shape and retention) and mass spectrometric ionisation. Disappearance rate of the study compound was estimated based on relative LC/MS peak areas (o min marked as 100%), and was used to calculate in vitro half-life and clearance (and in vivo extrapolation of hepatic clearance).

### Metabolic Stability in Mouse and Human Plasma

The compounds were incubated with plasma, and the collected samples were analyzed by UPLC/HR-MS to measure stability of the compounds.

Sample type: plasma;
Species: CD1-mouse (male), human (mixed gender);
Time points: 0, 20, 40, 60, 120 min;

### Concentration: 1 μM;

### Replicates: 2;

Spiking solvent: 50% DMSO ( 1/100 to incubation);
Quenching solvent: 2-fold volume of 100% ACN;
Control: propanthelin bromide (human, mouse)

The study compounds were incubated with plasma as specified above. The collected samples were stored at −20 C until thawed at room temperature, centrifuged and analyzed using UPLC/PDA with high resolution mass spectrometry (QE-Orbitrap-MS on DDI mode) to monitor substrate depletion and metabolite formation. The analytical method was optimised by using the parent compounds for optimum chromatographic properties (peak shape and retention) and mass spectrometric ionisation. Disappearance was based on relative LC/MS peak areas (0 min=100%) and will be used to calculate half-lives.

### Results

### Hepatic Toxicity

Cytotoxicity potency of test compounds SND118, SND121, SND122 and SND123 was assayed in human hepatocytes from pooled donor lot at three concentrations; 1, 10 and 100 μM. Cytotoxicity was assayed from medium samples after 24 h exposure period with test compounds by measuring membrane integrity (LDH leakage), coupled to fluorescent signal. In parallel, cell viability was assayed by the means of ATP content, indicating the metabolically competent cell activity. LDH and ATP assay kits were sourced from Promega. ATP content was measured based on luciferase catalysed reaction generating stable bioluminescent signal. Cytotoxic positive control chlorpromazine at 5-250 μM was used.

ATP content after incubation with SND118, SND121 and SND122 at 1 to 100 μM was 72-103%, 94-102%, 98-107% and 97-102%, respectively. Thus, the results do not suggest loss in viability by these compounds in the hepatocytes. Cell viability (ATP content) after incubation with SND123 at 1 to 100 μM was 1-108%. The results indicate dose-dependent loss in viability at higher doses as measured by ATP.

SND118, SND121 and SND122 resulted in 7-10%, 6-7%, 7-8% and 6-7% toxicity, respectively. The results do not indicate cytotoxicity by these compounds in the hepatocytes. Cytotoxicity (LDH) leakage after incubation with SND123 at 1 to 100 μM was 7-26% indicating dose-dependent, but low-level toxicity in the hepatocytes.

The results indicate some potential cytotoxicity at high concentrations for SND123, but not for the other compounds tested in this assay.

### Metabolic Stability

A series of SND derivatives have been tested for their stability in mouse and human plasma and intestinal and liver microsomes (IM and LM respectively). As summarized in Table 3, the compounds vary in their in vitro stability with SND122 being the less stable derivative. SND118 presents promising stability in plasma and human microsomes, which warrants further testing in vivo.

TABLE 3
Summary of plasma and microsome stability

| | T1/2 | T1/2 | T1/2 | T1/2 | T1/2 | T1/2 |
|---|---|---|---|---|---|---|
| | plasma | plasma | IM | IM | LM | LM |
| | Mouse | Human | mouse | human | mouse | human |
| Cpd | (min) | (min) | (min) | (min) | (min) | (min) |
| 118 | 789 | 789 | 27.6 | 30.8 | 23 | 18 |
| 121 | 789 | 789 | 316 | 6.4 | 5 | 144 |
| 122 | 9 | 62 | 2.5 | 1.8 | 2 | 5 |
| 123 | 789 | 789 | 132.8 | 106 | 6 | 56 |

1. A compound of formula (I): 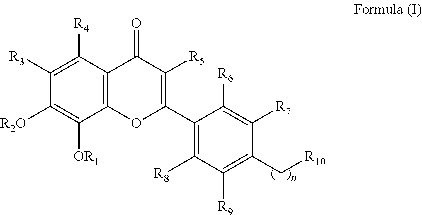
or a pharmaceutically acceptable multi-salt, solvate or prodrug thereof,
wherein:
R¹ and R², independently, are selected from H, hydroxyl protecting groups, —C₁₋₄ alkyl, —CH₂C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, and —OC(C≡CH)H₂; or R¹ and R² together form a C₁₋₄ alkylene group;
R³, R⁴, R⁵, R⁶, R⁷, R⁸, and R⁹, independently, are selected from H; halo; —CN; —NO₂; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and —OCOR^{β}; each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
R¹⁰ is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X rhodamine B X, rhodamine 6G X, rhodamine 19 X, or rhodamine 123 X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
and
n is an integer from 1 to 14.

2. (canceled)

3. The compound as claimed in claim 1, wherein R¹ and R² are independently selected from H and —C₁₋₄ alkyl CH—C(O)—R¹³, —SO₂R¹³, —C(O)SR¹³, —C(O)R¹³, —C(O)OR¹³, —C(O)NHR¹³, —C(O)N(R¹³)₂, —OCF₃, —OCHF₂, and —OC(C≡CH)H₂, or R¹ and R² together form a C₁₋₄ alkylene group.

4. The compound as claimed in claim 1, wherein R¹ and R² are H.

5. The compound as claimed in claim 1, wherein R³, R⁴, R⁵, R⁶, R⁷, R⁸, and R⁹ are independently selected from H; halo; —CN; —NO₂; —R^{β}; —OH; —OR^{β}; —NH₂; —NHR^{β}; —N(R^{β})²; —CHO; —COR^{β}; —COOH; —COOR^{β}; and —OCOR^{β}.

6. The compound as claimed in claim 1, wherein R³, R⁴, R⁵, R⁶, R⁷, R⁸, and R⁹ are H.

7. The compound as claimed in claim 1, wherein —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more halo, —OH, —NH₂, —CN, —NO₂, —C≡CH, —CHO, —CON(CH₃)₂ or oxo (═O) groups.

8. The compound as claimed in claim 1, wherein R¹⁰, is —[P(R¹¹)₃]X, —[N(R¹¹)₃]X, —[NHC(═NH₂)(NH₂)]X, —[NHC(═NH₂)NHC(═NH)(NH₂)]X, —[NHC(═NH)NHC(═NH₂)(NH₂)]X, rhodamine B X, or rhodamine 6G X, rhodamine 19 X, rhodamine 123 X, wherein each —R¹³ is independently selected from H, C₁-C₆ alkyl, C₂-C₆ alkenyl, C₃-C₁₄ aryl group, or C₃-C₁₄ aliphatic cyclic group.

9. The compound as claimed in claim 1, wherein, R¹⁰ is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups.

10. The compound as claimed in claim 1, wherein each R¹¹ group is the same.

11. The compound as claimed in claim 1, wherein the counter anion X is fluoride, chloride, bromide or iodide.

12. The compound as claimed in claim 1 having the following formula:

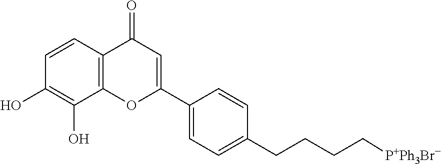

13. The compound as claimed in claim 1 having the following formula:

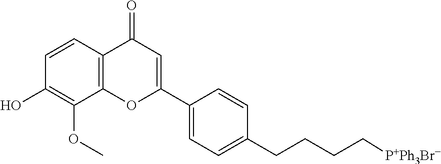

14. (canceled)

15. A pharmaceutical composition comprising the compound as defined in claim 1, and a pharmaceutically acceptable excipient.

16. (canceled)

17. (canceled)

18. (canceled)

19. A method of treatment or prevention of a disease, disorder or condition, the method comprising the step of administering an effective amount of the compound as defined in claim 1, or a pharmaceutically acceptable multi-salt, solvate or prodrug thereof, to thereby treat or prevent the disease, disorder or condition.

20. (canceled)

21. (canceled)

22. The compound as claimed in claim 1, wherein each R¹¹ is a phenyl group.

23. The method of treatment or prevention as claimed in claim 19, wherein the disease, disorder or condition is a central nervous system disease.
